(19) United States
(12) Patent Application Publication
(10) Pub. No.: US 2025/0345309 A1
(43) Pub. Date: Nov. 13, 2025

(54) IMMUNE HEALTH IMPROVERS

(71) Applicant: Amazentis SA, Lausanne (CH)

(72) Inventors: Christopher L. Rinsch, Lausanne (CH); Anurag Singh, Lausanne (CH); Florian Greten, Bad Homburg (DE); Dominic Denk, Fankfurt (DE)

(21) Appl. No.: 19/191,838

(22) Filed: Apr. 28, 2025

(30) Foreign Application Priority Data
Apr. 26, 2024 (GB) 2405936.2

(51) Int. Cl. A61K 31/37; A61K 9/00; A61K 9/06; A61K 45/06; A61P 37/02; C12N 5/00; C12N 5/0783; G01N 15/14; G01N 33/50

(52) U.S. Cl. CPC A61K 31/37; A61K 9/0053; A61K 9/06; A61K 45/06; A61P 37/02; C12N 5/0087; C12N 5/0636; G01N 15/14; G01N 33/505; G01N 33/5079; G01N 2500/10

(57) ABSTRACT

Disclosed are compositions that improve the functioning of the immune system in human subjects, in particular human subjects in which the immune system is of reduced effectiveness, or at risk of becoming less effective. Benefits relate in particular to the amelioration of immune aging, in particular, by the administration of urolithins, for example, urolithin A.

### RELATED APPLICATION

This application claims the benefit of priority to GB Patent Application No. 2405936.2, filed Apr. 26, 2024.

### BACKGROUND

The past century has seen a progressive demographic shift with an increase of life expectancy, resulting in an expected doubling of the population aged over 60 years by 2050. Yet, interventions to reliable extend healthspan, i.e. years of life without chronic conditions, are still lacking, thereby representing an urgent unmet need. Higher levels of systemic inflammation and immune dysfunction observed in the elderly favour T cell dysregulation, evidenced by cytokine storms in infectious disease recently observed in COVID-19 pandemic or blunted immune response to cancer, infections or vaccinations.

The immune system responds to harmful stimuli such as infections and cellular damage, providing a critical role in regulating inflammatory signalling required to combat exogenous infections or elicit an initial antitumor response at the presence of malignancy. A remarkable feature of the immune system is its capacity to form a long-term adaptive response enabling a rapid recall to future re-challenges termed immune memory, providing specific immunity that can span over decades. Yet, this principally advantageous plasticity actively specializing the immune system throughout life comes at the cost of reduced responsiveness to novel antigens which is highlighted by attenuated responses to vaccines and increased vulnerability to novel infectious diseases.

There is growing evidence from recent omics-based studies supporting the notion of aging-associated remodelling of immune populations (See, for example, Terekhova, M. et al. Single-cell atlas of healthy human blood unveils age-related loss of NKG2C+GZMB−CD8+ memory T cells and accumulation of type 2 memory T cells. Immunity 2023, 56, 2836-2854; and Alpert, A. et al. A clinically meaningful metric of immune age derived from high-dimensional longitudinal monitoring. Nature medicine 2019, 25, 487-495). Further, a correlation has been observed between a reduced lymphocyte count in the peripheral blood and age (Fagnoni, F. F. et al. Shortage of circulating naïve CD8+ T cells provides new insights on immunodeficiency in aging. Blood 2000, 95, 2860-2868).

Immunosenescence is the global remodelling of the immune system that is characterized by reduced thymic output, increased memory populations with concomitant reduction in naïve T cell populations. Notably, these changes cumulate into a sterile but chronic low-grade inflammatory state termed inflammaging (See Franceschi, C., Garagnani, P., Parini, P., Giuliani, C. & Santoro, A. Inflammaging: a new immune-metabolic viewpoint for age-related diseases. Nature reviews. Endocrinology 2018, 14, 576-590; and Furman, D. et al. Chronic inflammation in the etiology of disease across the life span. Naturemedicine 2019, 25, 1822-1832). This, in turn, increases the incidence of long-term inflammatory symptom and increases the incidence of heart disease and cancer. To date, no standard biomarkers for this have been established. However, a physician can make an assessment based on the sum of data available for the relevant markers.

To date, interventions aimed at improving immune health are restricted to generalized lifestyle interventions such as physical exercise or caloric restriction. For example, periodic cycles of a fasting mimicking diet (FMD) have been found in human subjects to be associated with reduced insulin resistance and other pre-diabetes markers, lower hepatic fat and increased lymphoid to myeloid ratio (See Brandhorst, et al. Fasting-mimicking diet causes hepatic and blood markers changes indicating reduced biological age and disease risk. Nat Commun 15, 1309 (2024)). The latter is an indicator of immune system age. Therapeutic approaches have been proposed. However, the development of these has been limited by long term safety concerns.

For example, Metformin has been proposed has a life-extending (geroprotector) drug. Promising results have been displayed in both invertebrate and vertebrate models, which indicate that metformin can target crucial mechanistic pathways involved in aging. However, several aspects of its use remain unclear, such as the influence of age, hormones, and dosage on its efficacy, and large scale clinical trials are yet to be conducted in humans (See M. G. Novelle et al. Cold Spring Harb Perspect Med 2016, 6 (3), a025932; and A. S. Kulkarni et al. Cell Metabolism 2020, 32, 15-30). Metformin has been used routinely as a first-line treatment for subjects with type 2 diabetes. In a non-diabetic subject, its use would be expected to be associated with unnecessary metabolic effects.

Rapamycin has been reported as extending lifespan in mice (See for example Harrison, D. E. et al. Rapamycin fed late in life extends lifespan in genetically heterogeneous mice. Nature 2009, 460, 392-395). As a central hub to metabolic function, mTOR centrally orchestrates T cell fate and inhibition of mTOR promotes memory T cell formation in mice under selective conditions, potentially benefiting immune response to murine infections and cancer. However, long term intake has been linked to spontaneous tumorigenesis in the liver. Its clinical use in humans as a potent immunosuppressant highlights that its incompletely understood immunomodulatory effects restrict routine use in healthy people without further safety studies.

A further approach to rejuvenating the immune system that has been proposed is the use of hematopoietic stem cells (HSCs), see for example Stankiewicz et al., Rebuilding and rebooting immunity with stem cells, Cell Stem Cell (2024), 31 (5), 597-616. This approach is currently experimental, and associated with high cost.

There thus remains a need for alternative treatments, which can attenuate the hallmarks of aging.

### SUMMARY OF THE INVENTION

The present invention relates to compositions that improve the functioning of the immune system in human subjects, in particular human subjects in which the immune system is of reduced effectiveness, or at risk of becoming less effective. The invention relates in particular to the amelioration of immune aging.

In a first embodiment of the invention there is provided a compound of formula (I), or a salt, prodrug, metabolite or derivative thereof;

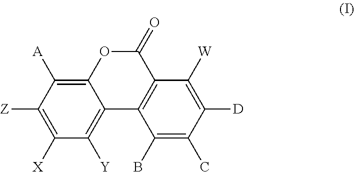

wherein:
A, B, C, D, W, X, Y and Z are each independently selected from H and OH;
   for use in ameliorating immune aging or improving immune health in a human subject.

The compound for use of the invention has been found to be surprisingly effective in reducing immune aging in human subjects. As described below in further detail, the current inventors have established that administration of the compound of the invention to human subjects profoundly alters immune phenotype and function, thereby ameliorating systemic inflammation.

The invention further provides the use of a compound of formula (I), or a salt, prodrug, metabolite or derivative thereof in the manufacture of a medicament for ameliorating immune aging or improving immune health in a human subject.

The invention further provides a method of ameliorating immune aging or improving immune health in a human subject comprising administering to the subject a compound of formula (I), or a salt, prodrug, metabolite or derivative thereof.

The invention further provides a composition comprising a compound of formula (I), or a salt, prodrug, metabolite or derivative thereof for use in ameliorating immune aging or improving immune health in a human subject.

The compounds and compositions of the invention also find utility in the treatment of non-human mammals.

### BRIEF DESCRIPTION OF THE FIGURES

FIG. 1A shows a clinical trial overview. Fifty (n=50) healthy participants were randomized (1:1) to either placebo softgels or 1000 mg UA per day. Blood was drawn at baseline, after seven days and 28 days to profile circulating immune cells.

FIG. 1B shows total lymphocyte counts of participants in the UA intervention arm (left panel) and placebo intervention arm (right panel). Changes from baseline measurement (do) and last study visit (d28) are shown. N=25 of matched samples, *p<0.05 by paired t test.

FIG. 1C shows total eosinophil counts of participants in the UA intervention arm (left panel) and placebo intervention arm (right panel). Changes from baseline measurement (d0) and last study visit (d28) are shown. N=25 of matched samples, *p<0.05 by paired t test.

FIG. 1D shows total count of neutrophiles in the UA group and the placebo group. Values for baseline visit and final study visit (d28) are shown. Data presented as a violi plot, n=25. All comparisons are not significant by paired t-test with intervention groups.

FIG. 1E shows total count of monocytes in the UA group and the placebo group. Values for baseline visit and final study visit (d28) are shown. Data presented as a violi plot, n=25. All comparisons are not significant by paired t-test with intervention groups

FIG. 1F shows total count of leukocytes in the UA group and the placebo group. Values for baseline visit and final study visit (d28) are shown. Data presented as a violi plot, n=25. All comparisons are not significant by paired t-test with intervention groups.

FIG. 2 shows representative gating results used to identify human immune subsets by spectral flow cytometry.

FIG. 3A shows an experimental overview. PBMCs from the UA intervention cohort (UA) and placebo cohort (P) were subjected to spectral flow cytometry. Immune profiling was performed with matched samples from all three study visits.

FIG. 3B shows changes in the T cell compartment assessed by spectral flow cytometry. Changes of ab CD3 cells depicted as absolute change in percentage of PBMCs are shown after 28 days of UA intake (U) or placebo (P). Data are ±95% CI, n=25 per group. All comparisons are not significant by two-sided t-test;

FIG. 3C shows changes in the T cell compartment assessed by spectral flow cytometry. Changes of ab CD8+ T cells depicted as absolute change in percentage of PBMCs are shown after 28 days of UA intake (U) or placebo (P). Data are ±95% CI, n=25 per group. All comparisons are not significant by two-sided t-test;

FIG. 3D shows changes in the T cell compartment assessed by spectral flow cytometry. Changes of ab CD4+ cells depicted as absolute change in percentage of PBMCs are shown after 28 days of UA intake (U) or placebo (P). Data are ±95% CI, n=25 per group. All comparisons are not significant by two-sided t-test;

FIG. 3E shows changes in CD8+ phenotype, CD8+ T_{SCM}.

FIG. 3F shows changes in CD8+ phenotype, CD8+ T_{N}.

FIG. 3G shows changes in CD8+ phenotype, CD8+ T_{EM}.

FIG. 3H shows changes CD8+ cells, Ki67+.

FIG. 3I shows changes CD8+ cells, TOX in CD.

FIG. 3J shows changes CD8+ cells, SA-β-gal.

FIG. 3K shows the amount of NK cells in PBMCs depicted as absolute change in percentage of total PBMCs.

FIG. 3L shows changes in the myeloid compartment, nonclassical monocytes.

FIG. 3M shows changes in the myeloid compartment, HLA-DR.

FIG. 4A shows the immune distribution analysis of unaltered lymphocyte subsets (UA cohort on the left panel and P cohort on the right panel). Percent change of total PBMCs (GD).

FIG. 4B shows the immune distribution analysis of unaltered lymphocyte subsets (UA cohort on the left panel and P cohort on the right panel). Percent change in leukocytes (CD8 Central Memory).

FIG. 4C shows the immune distribution analysis of unaltered lymphocyte subsets (UA cohort on the left panel and P cohort on the right panel). Percent change total PBMCs (CD8+ TVM).

FIG. 4D shows the immune distribution analysis of unaltered lymphocyte subsets (UA cohort on the left panel and P cohort on the right panel). Change in TEMRA.

FIG. 4E shows the immune distribution analysis of unaltered lymphocyte subsets (UA cohort on the left panel and P cohort on the right panel). Change in CD8 (% of leukocytes).

FIG. 4F shows the immune distribution analysis of unaltered lymphocyte subsets (UA cohort on the left panel and P cohort on the right panel). Change senescence marker p16.

FIG. 4G shows the immune distribution analysis of unaltered lymphocyte subsets (UA cohort on the left panel and P cohort on the right panel). Change senescence marker p18.

FIG. 4H shows the immune distribution analysis of unaltered lymphocyte subsets (UA cohort on the left panel and P cohort on the right panel). Change in CD4+ T_{SCM} population.

FIG. 4I shows the immune distribution analysis of unaltered lymphocyte subsets (UA cohort on the left panel and P cohort on the right panel). Change in CD4+ T_{N} population (PBMCs).

FIG. 4J shows the immune distribution analysis of unaltered lymphocyte subsets (UA cohort on the left panel and P cohort on the right panel). Change in CD4+ T_{CM} population (PBMCs).

FIG. 4K shows the immune distribution analysis of unaltered lymphocyte subsets (UA cohort on the left panel and P cohort on the right panel). Change in CD4+ T_{EM} population (PBMCs).

FIG. 4L shows the immune distribution analysis of unaltered lymphocyte subsets (UA cohort on the left panel and P cohort on the right panel). Change in CD4+ Th1 population (PBMCs).

FIG. 4M shows the immune distribution analysis of unaltered lymphocyte subsets (UA cohort on the left panel and P cohort on the right panel). Change in Gata3+ Th2 cells.

FIG. 4N shows the immune distribution analysis of unaltered lymphocyte subsets (UA cohort on the left panel and P cohort on the right panel). Change in regulatory CD4 cells.

FIG. 4O shows the immune distribution analysis of unaltered lymphocyte subsets (UA cohort on the left panel and P cohort on the right panel). Change in percentage of total B cells.

FIG. 4P shows the immune distribution analysis of unaltered lymphocyte subsets (UA cohort on the left panel and P cohort on the right panel). Change in percentage of Memory B cells.

FIG. 4Q shows the immune distribution analysis of unaltered lymphocyte subsets (UA cohort on the left panel and P cohort on the right panel). Change in percentage of Naive B cells.

FIG. 4R shows the immune distribution analysis of unaltered lymphocyte subsets (UA cohort on the left panel and P cohort on the right panel). Change in percentage of plasma cells among PBMCs.

FIG. 4S shows the immune distribution analysis of unaltered lymphocyte subsets (UA cohort on the left panel and P cohort on the right panel). Change in percentage of plasmablasts among PBMCs.

FIG. 5A shows the immune distribution analysis of additional unaltered immune populations (UA cohort on the left panel and P cohort on the right panel). Change in the expression of their inhibitory receptor NKG2A.

FIG. 5B shows the immune distribution analysis of additional unaltered immune populations (UA cohort on the left panel and P cohort on the right panel). Change in the expression of their inhibitory receptor KIR.

FIG. 5C shows the immune distribution analysis of additional unaltered immune populations (UA cohort on the left panel and P cohort on the right panel). Change in the expression of their inhibitory receptor KLRG1.

FIG. 5D shows the immune distribution analysis of additional unaltered immune populations (UA cohort on the left panel and P cohort on the right panel). Percent change in circulating DCs.

FIG. 5E shows the immune distribution analysis of additional unaltered immune populations (UA cohort on the left panel and P cohort on the right panel). Percent change in circulating ILCs.

FIG. 5F shows the immune distribution analysis of additional unaltered immune populations (UA cohort on the left panel and P cohort on the right panel). Percent change in classical monocytes.

FIG. 5G shows the immune distribution analysis of additional unaltered immune populations (UA cohort on the left panel and P cohort on the right panel). Percent change in intermediate monocytes.

FIG. 6A shows how the UA intake affects T cell mitochondria.

FIG. 6B shows changes in mitochondrial mass in CD8+ cells and CD4+ cells (UA cohort on the left panel and P cohort on the right panel).

FIG. 6D shows changes in mitochondrial mass in CD8+ cells and CD4+ cells (UA cohort on the left panel and P cohort on the right panel).

FIG. 6E shows division of CD8+ into T_{N}, T_{EM}, T_{CM} and T_{EFF} subset using the combination of CD45RA and CCR7.

FIG. 6F shows a comparison of mitochondrial mass in the T_{N}, T_{EM}, T_{CM} and T_{EFF} Subsets;

FIG. 6G shows the increase in mitochondrial mass in most CD8+ subsets, analyzed after 28 days of UA supplementation;

FIG. 6H shows expression of PGC-1α at baseline measurement and last study visit (d28) for the UA group and placebo group;

FIG. 6I shows the measurement of mitochondrial membrane potential in CD8+ cells at baseline measurement and last study visit (d28) for the UA group and placebo group.

FIG. 7A shows how the UA intake alters mitochondrial health. Mean Fluorescence Intensity (MFI) of PGC-1α among CD4+ cells at baseline and after 28 days in UA-treated and placebo-treated participants;

FIG. 7B shows how the UA intake alters mitochondrial health. Parkin expression within CD8+ cells at baseline and after 28 days in UA-treated and placebo-treated participants;

FIG. 7C shows how the UA intake alters mitochondrial health. Mitochondrial membrane potential assessed by MitoTrackerRed CMXROS staining in CD8+ subsets that were identified using CCR7 and CD45RA expression. Data at baseline and after 28 days are shown;

FIG. 7D shows how the UA intake alters mitochondrial health. Mitochondrial membrane potential assessed by MitoTrackerRed CMXROS staining in CD4+ cells at baseline and after 28 days in UA-treated and placebo-treated participants.

FIG. 8 shows how the application of SCENITH in the cohort recapitulates subset-specific metabolism in CD8+ cells for all participants at baseline.

FIG. 9A shows how UA intake potentiates mitochondrial capacity via glucose dependence, FAO&AAO capacity, mitochondrial dependence and glycolytic capacity (UA cohort on the left panel and P cohort on the right panel in each chart), for CD8+ cells.

FIG. 9B shows how UA intake potentiates mitochondrial capacity via glucose dependence, FAO&AAO capacity, mitochondrial dependence and glycolytic capacity (UA cohort on the left panel and P cohort on the right panel in each chart), for T_{N} cells.

FIG. 9C shows how UA intake potentiates mitochondrial capacity via glucose dependence, FAO&AAO capacity, mitochondrial dependence and glycolytic capacity (UA cohort on the left panel and P cohort on the right panel in each chart), for T_{EM} cells.

FIG. 9D shows how UA intake potentiates mitochondrial capacity via glucose dependence, FAO&AAO capacity, mitochondrial dependence and glycolytic capacity (UA cohort on the left panel and P cohort on the right panel in each chart), for T_{CM} cells.

FIG. 9E shows how UA intake potentiates mitochondrial capacity via glucose dependence, FAO&AAO capacity, mitochondrial dependence and glycolytic capacity (UA cohort on the left panel and P cohort on the right panel in each chart), for T_{EEF} cells.

FIG. 9F shows how UA intake potentiates mitochondrial capacity via glucose dependence, FAO&AAO capacity, mitochondrial dependence and glycolytic capacity (UA cohort on the left panel and P cohort on the right panel in each chart), for CD4+ cells.

FIG. 9G shows how UA intake potentiates mitochondrial capacity via glucose dependence, FAO&AAO capacity, mitochondrial dependence and glycolytic capacity (UA cohort on the left panel and P cohort on the right panel in each chart), for Classical Monocytes.

FIG. 9H shows how UA intake potentiates mitochondrial capacity via glucose dependence, FAO&AAO capacity, mitochondrial dependence and glycolytic capacity (UA cohort on the left panel and P cohort on the right panel in each chart), for Non-classical Monocytes.

FIG. 9I shows how UA intake potentiates mitochondrial capacity via glucose dependence, FAO&AAO capacity, mitochondrial dependence and glycolytic capacity (UA cohort on the left panel and P cohort on the right panel in each chart), for CD56^{dim}CD16^{hi} cells.

FIG. 9J shows how UA intake potentiates mitochondrial capacity via glucose dependence, FAO&AAO capacity, mitochondrial dependence and glycolytic capacity (UA cohort on the left panel and P cohort on the right panel in each chart), for B cells.

FIG. 10A shows how UA ameliorates inflammaging. Method of incubating PBMCs in the presence of αCD3/αCD28 stimulation beads.

FIG. 10B shows no difference between the two interventions, or baseline to final study (d28) visit, on T cell proliferation.

FIG. 10C shows no difference between the two interventions, or baseline to final study (d28) visit, on T cell proliferation.

FIG. 10D shows how UA ameliorates inflammaging. Preferential uptake of TCM and TEM phenotype in CD8+ cells after CD3 receptor engagement, Group A T cell simulation.

FIG. 10E shows how UA ameliorates inflammaging. Preferential uptake of TCM and TEM phenotype in CD8+ cells after CD3 receptor engagement, Group B stim.

FIG. 10F shows how UA ameliorates inflammaging. Supplementation with UA led to significantly lower levels of several plasma cytokines. No difference was observed for IL-6, IL-8, IL-17, IP-10, and IP-10 after UA or placebo intake, Group A cytokines.

FIG. 10G shows how UA ameliorates inflammaging. Supplementation with UA led to significantly lower levels of several plasma cytokines. No difference was observed for IL-6, IL-8, IL-17, IP-10, and IP-10 after UA or placebo intake, Group B cytokines.

FIG. 10H shows how UA ameliorates inflammaging. Antigen challenged CD8+ cells displayed reduced IL-4 production in UA-treated subjects when comparing baseline to final study (d28). No change was observed for the placebo participants.

FIG. 10I shows CD8+ expression of TNF-α remained unchanged in both UA and placebo participants at baseline and final study (d28) visit.

FIG. 10J shows CD8+ expression of IFN-γ remained unchanged in both UA and placebo participants at baseline and final study (d28) visit.

FIG. 10K shows how UA ameliorates inflammaging. Monocytes from UA-exposed participants exhibit enhanced phagocytosis of E. coli particles in comparison to the placebo cohort.

FIG. 10L shows how UA ameliorates inflammaging. Summary of the effects: the effects on T-cell stimulation, IL-4 production and E. coli uptake.

FIG. 11A shows the impact of UA intake on biological age clock with changes compared in UA (A, left) and Placebo (B, right) groups, using the PhenoAge 2nd generation clock.

FIG. 11B shows the impact of UA intake on biological age clock with changes compared in UA (A, left) and Placebo (B, right) groups, using the Hannum 1st generation clock.

FIG. 12 shows protein structures for CARS, TRUCKS and next generation molecules.

FIG. 13 shows the next generation protocol for the generation of CAR-T cells comprising the administration of urolithin A.

FIG. 14A shows representative gating strategy to identify MitotrackerGreen^{hi} cells within the CD3+ CD8+ population at baseline (d0) an after 28 days (d28) of UA and placebo supplementation, respectively.

FIG. 14B shows percentage of MitotrackerGreen^{hi} cells within CD8+ cells as identified in (a). Data are mean±95% CI, n=25 per group, *p<0.05 by repeated measures one-way ANOVA with Bonferroni's multiple comparison's test.

FIG. 14C shows percentage of MitotrackerGreen^{hi} cells within CD4+ cells. Data are mean±95% CI, n=25 per group. Repeated measures one-way ANOVA with Bonferroni's multiple comparison's test was used for statistical analysis.

FIG. 14D shows representative quadrant gating using CCR7 and CD45RA expression to identify TN, TCM, TEM and TEMRA within the CD8+ compartment.

FIG. 14E shows mitochondrial content of CD8+ subpopulations as depicted in (d). Percentage of MitotrackerGreen^{hi} cells within TN, TEM, TCM and TEMRA are shown. Data from all study participants at baseline (n=50) are shown.

FIG. 14F shows truncated violin plot depicting percentage of MitotrackerGreen^{hi} CD8+ subpopulations before (d0; unfilled violins to the left) and after UA supplementation (d28, color-filled violins to the right). Data from TN, TCM, TEM and TEMRA populations are shown as identified in (d). Mean (solid line) and 25th/75th quartiles (dotted lines) are shown. * p<0.05, ** p<0.01 and **** p≤0.0001 by paired t-test and Wilcoxon matched-pairs signed rank test with (for TEMRA) in CD8+ subpopulation studied. For non-significant comparisons, exact p-values are shown.

FIG. 14G shows expression of PGC-1α in CD8+ cells before (d0) and after (d28) UA and placebo supplementation, respectively. Assessed by flow cytometry, data shown as MFI. Representative histograms are shown. Data are mean±95% CI, n=12 per group, *p<0.05 by repeated measures one-way ANOVA with Bonferroni's multiple comparison's test.

FIG. 14H shows mitochondrial membrane potential (MMP) in CD8+ cells before (d0) and after (d28) UA and placebo supplementation, respectively. Assessed by MitotrackerCMXROS staining, data shown as MFI. Representative histograms are shown. Data are mean±95% CI, n=12 per group, *p<0.05 by repeated measures one-way ANOVA with Bonferroni's multiple comparison's test.

FIG. 15A shows UA induces metabolic reprogramming of the human immune system. Metabolic profiling of CD8+ cells using SCENITH. Truncated violin plots of glucose dependence are shown. Data shown from UA and placebo treated participants at baseline (d0) and after the 28 day supplementation period (d28) depicting percentage. Mean (solid line) and 25th/75th quartiles (dotted lines) are depicted. N=25 per group, ** p<0.01 by repeated measures one-way ANOVA with Bonferroni's multiple comparison's test.

FIG. 15B shows metabolic profiling of CD8+ cells using SCENITH. Truncated violin plots of fatty acid and amino acid oxidation capacity (FAO&AAO capacity) are shown. Data shown from UA and placebo treated participants at baseline (do) and after the 28 day supplementation period (d28) depicting percentage. Mean (solid line) and 25th/75th quartiles (dotted lines) are depicted. N=25 per group, ** p<0.01 by repeated measures one-way ANOVA with Bonferroni's multiple comparison's test.

FIG. 15C shows metabolic profiling of CD8+ cells using SCENITH. Truncated violin plots of mitochondrial dependence are shown. Data shown from UA and placebo treated participants at baseline (d0) and after the 28 day supplementation period (d28) depicting percentage. Mean (solid line) and 25th/75th quartiles (dotted lines) are depicted. N=25 per group, ** p<0.01 by repeated measures one-way ANOVA with Bonferroni's multiple comparison's test.

FIG. 15D shows metabolic profiling of CD8+ cells using SCENITH. Truncated violin plots of glycolytic capacity are shown. Data shown from UA and placebo treated participants at baseline (d0) and after the 28 day supplementation period (d28) depicting percentage. Mean (solid line) and 25th/75th quartiles (dotted lines) are depicted. N=25 per group, ** p<0.01 by repeated measures one-way ANOVA with Bonferroni's multiple comparison's test.

FIG. 15E shows Truncated violin plots of fatty acid and amino acid oxidation (FAO&AAO) from several immune populations as assessed by SCENITH. Data from CD4+ cells are shown from UA and placebo treated participants at baseline (d0) and after the 28-day supplementation period (d28). Mean (solid line) and 25th/75th quartiles (dotted lines) are depicted. N=25 per group, *p<0.05 by repeated measures one-way ANOVA with Bonferroni's multiple comparison's test.

FIG. 15F shows Truncated violin plots of fatty acid and amino acid oxidation (FAO&AAO) from several immune populations as assessed by SCENITH. Data from CD4+ cells B cells are shown from UA and placebo treated participants at baseline (d0) and after the 28-day supplementation period (d28). Mean (solid line) and 25th/75th quartiles (dotted lines) are depicted. N=25 per group, *p<0.05 by repeated measures one-way ANOVA with Bonferroni's multiple comparison's test.

FIG. 15G shows Truncated violin plots of fatty acid and amino acid oxidation (FAO&AAO) from several immune populations as assessed by SCENITH. Data from NK cells are shown from UA and placebo treated participants at baseline (d0) and after the 28-day supplementation period (d28). Mean (solid line) and 25th/75th quartiles (dotted lines) are depicted. N=25 per group, *p<0.05 by repeated measures one-way ANOVA with Friedman test with Dunn's multiple comparisons test.

FIG. 15H shows Truncated violin plots of fatty acid and amino acid oxidation (FAO&AAO) from several immune populations as assessed by SCENITH. Data from nonclassical monocytes are shown from UA and placebo treated participants at baseline (d0) and after the 28-day supplementation period (d28). Mean (solid line) and 25th/75th quartiles (dotted lines) are depicted. N=25 per group, *p<0.05 by repeated measures one-way ANOVA with Friedman test with Dunn's multiple comparisons test.

FIG. 16A shows Unsupervised Uniform Manifold Approximation and Projection (UMAP) visualization of PBMCs with clusters identified by unsupervised hierarchical clustering.

FIG. 16B shows Volcano plot of differentially expressed genes within CD8+ cells in UA treated participants vs. placebo after the 28-day intervention period. Red dots represent genes expressed at higher levels in CD8+ cells from UA-treated participants, while blue dots represent genes with lower expression levels compared to baseline visit. Genes with log 2fc≥0.1 and p<0.05 were considered significant.

FIG. 16C shows an Ingenuity pathway Analysis (IPA) of CD8+ cells, displaying activated canonical pathways in CD8+ cells of UA treated participants. Ranking was performed based on log 10 p-value. Z-scores of affected pathways are shown.

FIG. 16D shows an Ingenuity pathway Analysis (IPA) of CD8+ cells, displaying inhibited canonical pathways in CD8+ cells of UA treated participants. Ranking was performed based on log 10 p-value. Z-scores of affected pathways are shown.

FIG. 16E shows a biological upstream regulator analysis of CD8+ cells from UA-treated participants after the intervention period. Predicted inhibited upstream regulators as established by IPA are shown, ranked based on-log 10 p-value. Respective inhibition z-scores are shown.

FIG. 16F shows a Heatmap showing the differential expression of markers of interest among NK cells. Left shows up-regulated IEGs. The color scale is based on log 2fc-scaled gene expression compared to the baseline visit.

FIG. 16G shows a Dot plot for Gene Ontology (GO) term enrichment analysis of NK cells. Top upregulated terms and suppressed terms among BP (biological processes) and CC (cellular components) domains are shown. Ranking was performed on enrichment score. Genes with log 2fc≥0.1 and p<0.05 were considered.

FIG. 16H shows a Volcano plot of differentially expressed genes within monocytes in UA treated participants vs. placebo. Red dots represent genes expressed at higher levels in monocytes from UA-treated participants, while blue dots represent genes with lower expression levels. Genes with log 2fc≥0.1 and p<0.05 were considered significant.

FIG. 16I shows a canonical pathway analysis of monocytes. Shows top 10 activated (red) and inhibited (blue) pathways in monocytes of UA-treated participants compared to the baseline measurement. Ranking was performed based on-log 10 adjusted p-value. Z-scores are shown.

FIG. 16J shows an upstream regulator analysis of monocytes using IPA. Top 6 predicted activated (red) and inhibited (blue) upstream regulators are shown. Ranking was performed based on Z-score.−log 10 p-values are shown.

FIG. 17A shows Plasma cytokine measurements. Fold change compared to baseline plasma levels of the indicated cytokines of UA (treated participants are shown. Data are mean±95% CI, n=14-20 of matched samples per cytokine. * p<0.05, ** p<0.01 by paired t-testing or Wilcoxon matched-pairs signed rank test.

FIG. 17B shows Plasma cytokine measurements. Fold change compared to baseline plasma levels of the indicated cytokines of placebo treated participants are shown. Data are mean±95% CI, n=14-20 of matched samples per cytokine. * p<0.05, ** p<0.01 by paired t-testing or Wilcoxon matched-pairs signed rank test.

FIG. 17C shows a PBMC stimulation overview. Thawed PBMCs from UA and placebo treated individuals were stimulated with αCD3/αCD28 beads for a total duration of 4 days, followed by functional readouts.

FIG. 17D shows the percentage of IL-4hi cells within CD8+ cells after four days of PBMC stimulation as depicted in (a). Data are mean±95% CI, n=25 per group, ** p<0.01 by Friedman test with Dunn's multiple comparisons test. For non-significant comparisons, exact p-values are shown.

FIG. 17E shows Expression of TNF-α in CD8+ cells before (d0) and after (d28) UA and placebo supplementation, respectively. Cells were stimulated as depicted in (a). Assessed by flow cytometry, data shown as MFI. Representative histograms are shown. Data are mean±95% CI, n=25 per group, ** p<0.01 by Friedman test with Dunn's multiple comparisons test. For non-significant comparisons, exact p-values are shown.

FIG. 17F shows E. coli uptake of monocytes from UA and placebo-treated participants before (d0) and after the supplementation period (d28), respectively. Data are mean±95% CI, n=25 per group, **** p≤0.0001 by repeated measures one-way ANOVA with Bonferroni's multiple comparison's test. For non-significant comparisons, exact p-values are shown.

FIG. 18A shows a Violin plot displaying the average number of genes per cell. Data from every analyzed sample shown (n=5 per treatment, matched do and d28 samples).

FIG. 18B shows Gene Ontology (GO) term enrichment analysis of CD8+ cells after 28 days of UA intake using the web-based tool Enrichr. Top upregulated terms and suppressed terms among the “cellular components” domain are shown. Ranking was performed based on adjusted-log 10 p-value. Genes with log 2fc≥0.1 and p<0.05 were considered for input.

FIG. 18C shows a Volcano plot of differentially expressed genes within CD8+ TEM in UA treated participants vs. placebo after the 28-day intervention period. Red dots represent genes expressed at higher levels in T_{EM} from UA-treated participants, while blue dots represent genes with lower expression levels. Genes with log 2fc≥0.1 and p<0.05 were considered significant.

FIG. 18D shows Gene Ontology (GO) term enrichment analysis of CD8+ TEM cells after UA intake. Top upregulated terms and suppressed terms among CC (cellular components) and MF (molecular functions) domains are shown. Ranking was performed on adjusted-log 10 p-value. Genes with log 2fc≥0.1 and p<0.05 were considered for input.

FIG. 18E shows a Volcano plot of differentially expressed genes within CD8+ T_{N} in UA treated participants vs. placebo after the 28-day intervention period. Red dots represent genes expressed at higher levels in TN from UA-treated participants, while blue dots represent genes with lower expression levels. Genes with log 2fc≥0.1 and p<0.05 were considered significant.

FIG. 18F shows a Volcano plot of differentially expressed genes within B cells in UA treated participants vs. placebo after the 28-day intervention period. Red dots represent genes expressed at higher levels in B cells from UA-treated participants, while blue dots represent genes with lower expression levels. Genes with log 2fc≥0.1 and p<0.05 were considered significant.

FIG. 18G shows a Canonical pathway analysis of B cells. Shows top 5 activated pathways in B cells of UA-treated participants compared to the baseline measurement. Ranking was performed based on-log 10 adjusted p-value. Z-scores are shown.

FIG. 18H shows a Canonical pathway analysis of B cells. Shows top 5 inhibited pathways in B cells of UA-treated participants compared to the baseline measurement. Ranking was performed based on-log 10 adjusted p-value. Z-scores are shown.

FIG. 18I shows a Volcano plot of differentially expressed genes within CD4+ cells in UA treated participants vs. placebo after the 28-day intervention period. Red dots represent genes expressed at higher levels in CD4 cells from UA-treated participants, while blue dots represent genes with lower expression levels. Genes with log 2fc≥0.1 and p<0.05 were considered significant.

FIG. 18J shows a Canonical pathway analysis of CD4+ cells. Shows top 5 activated (red; j) and inhibited (blue; k) pathways in CD4 cells of UA-treated participants compared to the baseline measurement. Ranking was performed based on-log 10 adjusted p-value. Z-scores are shown.

FIG. 18K shows a Canonical pathway analysis of CD4+ cells. Shows top 5 activated (red; j) and inhibited (blue; k) pathways in CD4 cells of UA-treated participants compared to the baseline measurement. Ranking was performed based on-log 10 adjusted p-value. Z-scores are shown.

### DETAILED DESCRIPTION OF THE INVENTION

As described above, the invention provides a compound of formula (I), or a salt, prodrug, metabolite or derivative thereof;

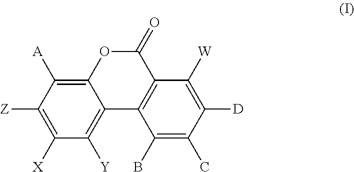

wherein:
A, B, C, D, W, X, Y and Z are each independently selected from H and OH;
   for use in ameliorating immune aging or improving immune health in a human subject.

The current inventors have established for the first time that the immune cell populations in human subjects are remodeled towards a younger phenotype upon administration of a urolithin (Urolithin A). In particular, it has been found that the ratio of CD8+ (T_{N}) T cells to CD8+ (T_{EM}) T cells is shifted towards there being a higher proportion of naïve CD8+ T cells (T_{N}) upon administration of a urolithin (Urolithin A).

In more detail, the current inventors have established in a clinical trial in human subjects that Urolithin A ameliorates immune aging and improves immune health: When human subjects took 1 g per day of Urolithin A for 28 days, subjects in the treatment group displayed significantly more circulating lymphocytes and eosinophils at the end of the treatment period compared to baseline levels, a change that was not present in the placebo group. Significantly and as described in further detail below, the proportion of naïve CD8+ T cells (T_{N}) among total PBMCs was increased after Urolithin A intake compared to placebo and the proportion of CD8+ effector memory cells (T_{EM}) were reduced. That is to say that the ratio of CD8+ (T_{N}) T cells to CD8+ (T_{EM}) T cells is shifted towards there being a higher proportion of naïve CD8+ T cells (T_{N}). That change was not seen in the placebo group.

Furthermore, the proportion of NK cells among total PBMCs was increased after Urolithin A intake compared to placebo. That change was not seen in the placebo group. NK cells are highly significant cells in the immune system. As effector lymphocytes of the innate immune system they are key to controlling several types of tumors and microbial infections. A low NK cell count is associated with poor lifespan.

Other immune populations, such as monocytes, total leukocytes and neutrophiles did not show a marked change in either group.

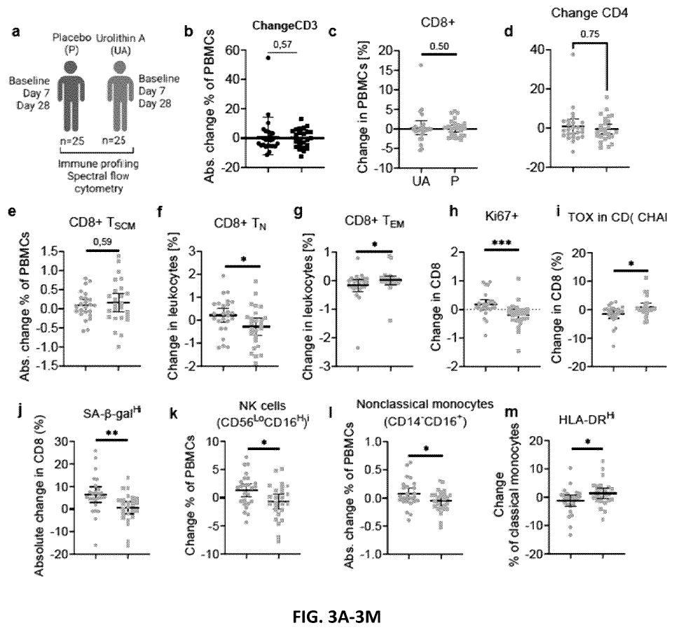

Broad spectral cytometry was carried out to characterize the immune phenotype upon Urolithin A intake. Over 30 markers were investigated, comprising immune cell surface markers and transcription factors. No differences were found in the percentage of total αβCD3+, CD8+, CD4+ T cells or circulating γδ T cells among total PBMCs analysed. However, marked changes within CD8+ subpopulations were found: The proportion of naïve T cells (T_{N}) among total PBMCs was increased after Urolithin A intake compared to placebo. Effector memory cells (T_{EM}), were concomitantly reduced in the subjects treated with Urolithin A. In the elderly, the proportion of naïve T cells (T_{N}) generally reduces with age and the proportion of effector memory cells (T_{EM}) generally increases with age (potentially by over 50% in each case-see FIG. 3a in Alpert et al (Alpert et al., A clinically meaningful metric of immune age derived from high-dimensional longitudinal monitoring. Nature medicine 25, 487-495; 10.1038/s41591-019-0381-y (2019)). In the group of human subjects treated with Urolithin A, the opposite of this was observed. That is to say that the aging phenotype was seen to be reversed.

The compound for use in accordance with the invention thus finds particular use in a human subject that is over 50 years of age, for example over 55 years or age, for example over 60 years of age. For example, the compound for use in accordance with the invention finds particular use in a human subject that is over 65 years of age, for example over 70 years or age, for example over 75 years of age, for example over 80 years or age, for example over 85 years of age. In a subject who has had certain diseases (for example cancer), the onset of significant immune aging can be younger. That is to say that, in some groups of subjects, the invention finds particular use in a human subject that is over 20 years of age for example over 25 years of age, for example over 30 years of age, for example over 35 years of age, for example over 40 years of age, for example over 45 years of age.

Other CD8+ subsets, such as central memory cells (T_{CM}), TEMRA or recently characterized “virtual memory” cells (T_{VM}) that arise with a memory-like phenotype without prior foreign antigen challenge, remained unaffected in both cohorts.

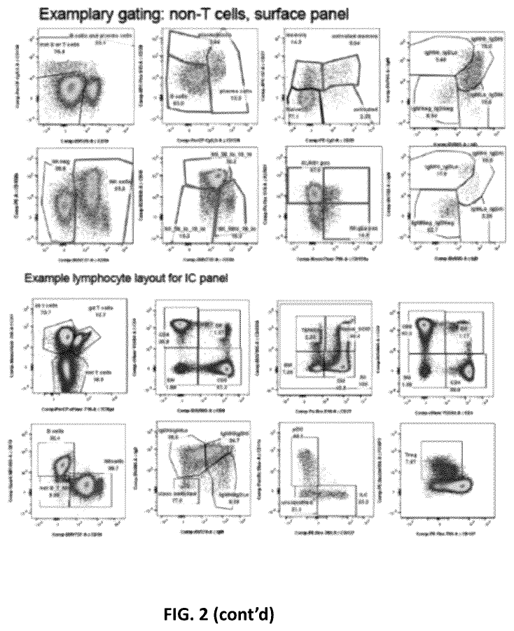

It was further observed that CD8+ cells in the Urolithin A group displayed more Ki67 (FIG. 2H) after the completion of the 28 day treatment with Urolithin A. Ki67 is a marker of cellular proliferation and T-cell reinvigoration that predicts pathological complete response to immune checkpoint blockade in patients with triple-negative breast cancer. A small reduction in TOX expression was also observed. TOX is considered the master regulator of T cell exhaustion that marks aging-associated T cells (Taa) and promotes CD8+ T cell dysfunction in cancer. PD-1 expression was unaltered in both groups.

No differences were observed in CD4+ T_{SCM}, T_{N}, T_{EM} or T_{CM} populations. There were also no changes in CD4+ Th1 cells (marked by T-bet expression), Gata3+ Th2 cells, FoxP3+ T_{reg} or circulating T follicular helper cells (Tfh1, identified via Bcl-6). Urolithin A intake did therefore not affect the phenotype of CD4+ cells in the human subjects and there were also no changes in percentage of total B cells, plasma cells or plasmablasts among PBMCs or specific B cell subsets between the two intervention groups.

In the investigation of other cell groups: CD56^{dim}CD16^{bright} NK (Natural Killer) cells, the most common NK subset in the blood, were markedly expanded among total PBMCs in the Urolithin A group. No change was observed in the expression of their inhibitory receptors such as NKG2A, KIR or KLRG1. Circulating DCs and ILCs also did not change upon either intervention.

When further interrogating monocyte populations, it was found that nonclassical monocytes (defined as CD14^{lo}CD16^{hi} cells) were increased among total PBMCs after the 28 day treatment period compared to placebo, whereas intermediate monocytes

(CD14hiCD16hi) and classical monocytes (CD14^{lo}CD16^{hi}) did not undergo change between the two groups. The classical monocytes exhibited less HLA-DR in subjects in the Urolithin A treatment group at the final study visit, indicative of a less inflammatory phenotype.

Altogether, the administration of Urolithin A deeply altered the CD8+ phenotype into a more naïve-like, less exhausted global state. The Urolithin A did not have effects on the CD4+ Tcell population.

Urolithin A has been suggested previously to provide some benefits in immune health. For example, it was shown to bring about enrichment of T-memory stem cells ex vivo (WO2023/161453) when it was present during stimulation with aCD3/aCD28 beads. However, these suggestions have not indicated that the extensive shift in the immune cell population towards a younger phenotype would be possible, with, for example an increased proportion of naïve CD8+ T cells (T_{N}) and/or the increased proportion of NK cells that has been found.

For example, the human subject is one with a ratio of naïve CD8+ T cells to effector memory CD8+ T cells that is biased towards memory CD8+ T cells. For example, the amelioration of immune aging or improvement of immune health in the human subject is an amelioration of inflammaging. For example, the human subject is one displaying signs or symptoms of inflammaging.

Immune aging is characterized by numerous changes to the immune system. Many of the changes have been summarised by Alpert et al. (see Alpert, A. et al. A clinically meaningful metric of immune age derived from high-dimensional longitudinal monitoring. Nature medicine 25, 487-495; 10.1038/s41591-019-0381-y (2019)). For example, immune aging can be considered to be present when there are at least one, for example at least two, for example three of the following, for example at least 4 of the following, for example all of the following in the subject:
Decline in lymphocyte count
Decline in naïve CD8+ T cell population
Decline in NK cell population
Decline in mitochondrial function in immune cells
Increase in inflammation

That is to say that the invention provides a compound for use in accordance with the invention wherein the amelioration of immune aging or improvement in immune health is one or more of:
an increase in the ratio of CD8+ (T_{N}) T cells to CD8+ (T_{EM}) T cells so that the proportion of naïve CD8+ T cells is increased;
an increase in the proportion of CD56^{dim}CD16^{bright} NK (Natural Killer) cells in the total PBMC population;
an increase in mitochondrial function in immune cells;
a decrease in inflammatory markers;
an increase in circulating lymphocyte count.

As the invention brings about a broad shift in the immune cell population, it is effective to improve bring about at least three, for example at least 4, and especially all 5 of the measures mentioned above.

In particular, the invention provides a compound of formula (I), or a salt, prodrug, metabolite or derivative thereof for increasing the level of NK cells in the subject, for example for increasing in the proportion of CD56^{dim}CD16^{bright} NK (Natural Killer) cells in the total PBMC population. The invention provides a method of increasing the level of NK cells in the subject, for example for increasing in the proportion of CD56^{dim}CD16^{bright} NK (Natural Killer) cells in the total PBMC population.

The ratio of CD8+ (T_{N}) T cells to CD8+ (T_{EM}) T cells can be assessed by cellular analysis of a PBMC sample taken from the subject, followed by calculation of the proportion of cells of the two types that are present.

The proportion of CD56^{dim}CD16^{bright} NK (Natural Killer) cells in the total PBMC population can be assessed by cellular analysis of a PBMC sample taken from the subject.

The level of mitochondrial function in immune cells can be established using the method referred to as SCENITH and described in further detail in Argüello et al., SCENITH: A flow cytometry-based method to functionally profile energy metabolism with single cell resolution, 2020, Cell Metabolism, 32, 1063-1075).

The level of inflammatory markers can be established by measuring the concentration of the markers in a blood sample taken from the subject. Examples of suitable markers include C-reactive protein (CRP), IL-6, IL-8, IL-1β, IL-2, IL-4 and TNFα, IFNγ and IL-10.

The circulating lymphocyte count can be established by cellular analysis of a blood sample taken from the subject.

In a further embodiment, immune aging can be assessed using the IMM-AGE score, as described in Alpert et al (2019). A related measure is described in WO 2019/215740. As set out therein, the immunological age of a subject may be assessed by a method comprising:
a. measuring relative abundance of at least 3 immune cell populations in a blood sample from said subject; and
b. determining an immunological age of said subject by at least one of:
   i. comparing said measurement of relative abundance of immune cell populations to a dataset of measurements of immune cell population relative abundance in subjects with predetermined immunological ages; and
   ii. combining said measurement of relative abundances with data of measurements of relative abundance from at least 19 other subjects to produce a database, calculating from said database a trajectory for all at least 20 subjects based on said measurements of relative abundance; thereby determining the immunological age of said subject.

The 3 immune cell populations are preferably selected from the group consisting of: naive CD8+ T cells, effector CD8+ T cells, CD28− CD8+ T cells, B cells, CXCR5+ CD4+ T cells, CD161− CD45RA+ T regulator cells, naive CD4+ T cells, CXCR5+ CD8+ T cells, HLADR− CD38+ CD4+ T cells, Th17 CXCR5− CD4+ T cells, T cells, CD85j+ CD8+ T cells, CD57+CD8+ T cells, Th2 non-TFH CD4+ T cells, PD1+ CD8+ T cells, effector memory CD4+ T cells, CD27+ CD8+ T cells, lymphocytes, central memory CD4+ T cells, natural killer (NK) cells, monocytes, Thl TFH CD4+ T cells, CD8+ T cells, CXCR3− CCR6− CXCR5+ CD8+ T cells, Th2 TFH CD4+ T cells, plasmablasts, and CD94+NK cells.

Accordingly, the invention provides a compounds for use wherein the human subject is one with an IMM-AGE score indicating a higher mortality than the average for a subject of the same age. The invention also provides a method of reducing the IMM-AGE score in a subject comprising administering a compound of Formula (I) to the subject. As discussed above, the IMM-AGE algorithm integrates the main elements that indicate an individual's immune system age, or degree of immunesenscence. The IMM-AGE metric provides a value from 0 to 1. The higher the IMM-AGE score, the higher is the individual's mortality risk. A healthy individual generally has a score of 0.5. The invention finds use in reducing the IMM-AGE score in an individual who has a higher than desired IMM-AGE score, for example an IMM-AGE score of over 0.5.

To functionally characterize the human immune system after systemic Urolithin A supplementation, the proliferative capacity and cytokine secretion by T cells was investigated. PBMCs removed from the Urolithin A and placebo treated groups were incubated in the presence of αCD3/αCD28 stimulation beads for a total of four days.

Inflammaging is characterized by increased levels of proinflammatory cytokines (See Furman, D. et al. Chronic inflammation in the etiology of disease across the life span. Nature medicine 2019, 25, 1822-1832). Considering the changes brought about UA on the cells of the immune system and the observation that aging-associated metabolic failure profoundly contributes to the phenotype of inflammaging, it was investigated whether circulating plasma levels of relevant cytokines are affected by UA supplementation.

It was observed that administration of UA in a period of 28 days reduced the levels of the cytokines TNF-α, IL-1β, IFN-γ, IL-2, and IL-10 found in the plasma, a change that can be characterised as anti-inflammatory on a systemic level. No difference was found for IL-6, IL-8, IL-17, interferon-gamma induced protein (IP-10) and monocyte chemoattractant protein-1 (MCP-1) in the UA or placebo group.

A recent single-cell atlas of healthy human blood has indicated an aging-associated bias towards type 2 immunity that is uncovered in healthy subjects upon antigen challenge, but not at steady-state. We therefore next focused on cytokine expression of stimulated T cells to detect potential differences in the context of immune response which could constitute a potential predisposition to conditions that may only manifest when specifically triggered. Intriguingly, antigen challenged CD8+ cells of UA-treated subjects displayed reduced IL-4 production, whereas placebo intake did not elicit any changes. Despite the pronounced anti-inflammatory signature observed in the plasma, CD8+ expression of IFN-γ or TNF-α remained unchanged when T cells were stimulated.

Taken together, UA treatment resulted in reduced circulating proinflammatory cytokines at steady-state and reduced IL-4 expression in the context of an antigen-provoked T cell response.

The immunosenescent phenotype is considered responsible for an increased susceptibility to infections. The ability of monocyte cells to phagocytose gram-negative bacteria was investigated for the two treatment groups. It was found that monocytes from UA-exposed participants exhibited significantly enhanced phagocytosis of E. coli particles ex vivo when compared with the placebo cohort. This demonstrates that the Urolithin A brought about superior uptake and clearance of gram-negative bacteria.

Collectively, the data supports that UA supplementation results in changes in immune function that carries systemic consequences and will lead to improved outcomes in the subject.

That is to say that the invention is effective for use in the treatment or prevention of a bacterial infection or a viral infection.

The invention finds particular use in a human subject who has been identified as having one or more of:
a ratio of CD8+ (T_{N}) T cells to CD8+ (T_{EM}) T cells that is lower than needed for strong immune health;
a proportion of CD56^{dim}CD16^{bright} NK (Natural Killer) cells is lower than needed for strong immune health;
mitochondrial function in immune cells that is lower than needed for strong immune health;
inflammatory markers indicative of significant inflammaging;
a circulating lymphocyte count that is lower than needed for strong immune health.

As the invention brings about a broad shift in the immune cell population, the human subject is most preferably one who has been identified as having at least 3, for example at least 4, and especially all 5 of the measures mentioned above.

An important feature of the current invention is that the positive effects in the amelioration of immune aging (and other conditions mentioned herein) are brought about safely—with no observed adverse effects on the subjects. In the study described herein, 25 human subjects took 1 g of Urolithin A per day for 28 days. The adverse events noted during the study were all mild, at the same level in the treatment and placebo groups and at a level that would be expected in the general population. Previous studies of the administration of Urolithin A to human subjects for other purposes have also established that that compound is safe for long term use in humans.

The compound for use according to the invention is thus especially advantageous in a human subject is one in whom it is important to avoid systemic side effects. For example, the compound for use may be employed in a use wherein the human subject is one with reduced liver function (for example mild, moderate or severe liver function impairment) or reduced kidney function (for example mild, moderate or severe kidney function impairment). For example, the compound for use may be employed in a use wherein the human subject is one with a compromised immune system, for example as a result of a medical procedure, for example as a result of a stem cell ablation treatment.

The invention further provides a compound of formula (I), or a salt, prodrug, metabolite or derivative thereof;

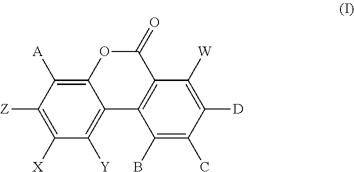

wherein:
A, B, C, D, W, X, Y and Z are each independently selected from H and OH for use in reducing T-cell aging.

In a further embodiment, the invention further provides a compound of formula (I), or a salt, prodrug, metabolite or derivative thereof for reducing natural killer cell aging.

In a further embodiment, the invention further provides a compound of formula (I), or a salt, prodrug, metabolite or derivative thereof for promoting T-cell regeneration.

In a further embodiment, the invention further provides a compound of formula (I), or a salt, prodrug, metabolite or derivative thereof for promoting natural killer cell regeneration.

The invention further provides a compound of formula (I), or a salt, prodrug, metabolite or derivative thereof;

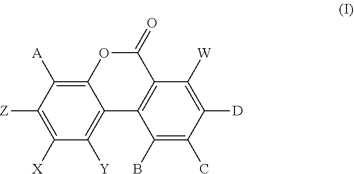

wherein:
A, B, C, D, W, X, Y and Z are each independently selected from H and OH for use in reducing inflammation, for example, systemic inflammation.

According to a further embodiment of the invention there is provided a compound of formula (I), or a salt, prodrug, metabolite or derivative thereof; for use in reducing inflammation, for example, systemic inflammation, wherein the compound of formula (I), for example urolithin A is administered a dose in the range of about 500 mg and about 1500 mg daily, for example, as a single dose, for example, in the range of about 500 mg to about 1200 mg, for example, in the range 700 mg to about 1200 mg, for example, in the range about 800 mg to about 1200 mg, for example, in the range about 900 mg to 1100 mg, for example, about 500 mg, about 600 mg, about 700 mg, about 800 mg, about 900 mg, about 1000 mg, about 1100 mg about 1200 mg, about 1300 mg, about 1400 mg or about 1500 mg.

### Biological Age

Immune health is central to overall health and strong immune health is correlated with low mortality and lower biological age. The invention therefore provides a compound of formula (I), or a salt, prodrug, metabolite or derivative thereof;

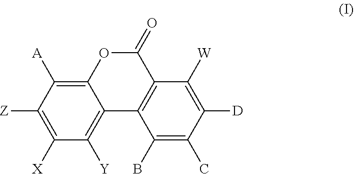

wherein:
A, B, C, D, W, X, Y and Z are each independently selected from H and OH for use in reducing biological age of a human subject, for example as assessed based the degree of DNA methylation in the subject, for example as based on the degree of methylation of DNA in a sample of circulating PBMCs obtained from the subject.

The DNA methylation can be assessed using established methods, including methods that are commercially available, for example, from TruDiagnostic.

In embodiments, the biological age is biological immune age or a biological age specific to a target organ, for example skin biological age, brain biological age or muscle biological age. Biological age of a particular target organ can be assessed by analysing the degree of methylation of DNA obtained from the organ in question. For example, it has been established that skin-specific methylome analysis can be used to find the biological age of the skin; see for example Boroni, M., Zonari, A., Reis de Oliveira, C. et al. Highly accurate skin-specific methylome analysis algorithm as a platform to screen and validate therapeutics for healthy aging. Clin Epigenet 12, 105 (2020).

Aging is often marked by the establishment of global hypomethylation and regions of CpG island hypermethylation. The invention provides a method of reducing the degree of hypomethylation and regions of CpG island hypermethylation in a human subject.

The improvement in biological age is, for example, achieved in addition to the amelioration of immune aging and improvement of immune health.

In a further embodiment, there is provided a compound of formula (I), or a salt, prodrug, metabolite or derivative thereof;

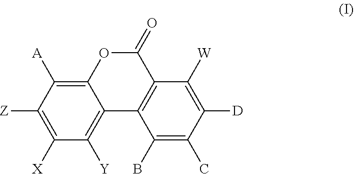

wherein:
A, B, C, D, W, X, Y and Z are each independently selected from H and OH, (for example, urolithin A) for use in reducing biological age in immune cells, for example, biological age of peripheral blood mononuclear cells.

### Immune Senescence/Immune Revival

The invention further provides a compound of formula (I), or a salt, prodrug, metabolite or derivative thereof;

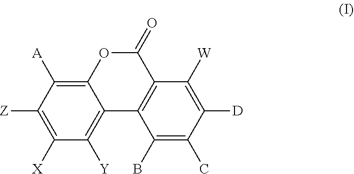

wherein:
A, B, C, D, W, X, Y and Z are each independently selected from H and OH, (for example, urolithin A) for reducing immune senescence.

The invention further provides a compound of formula (I), or a salt, prodrug, metabolite or derivative thereof;

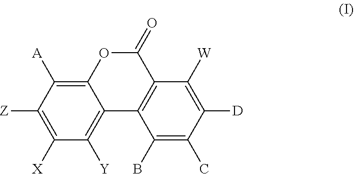

wherein:
A, B, C, D, W, X, Y and Z are each independently selected from H and OH, for example urolitihin A) for promoting immune revival.

The invention further provides a compound of formula (I), or a salt, prodrug, metabolite or derivative thereof;

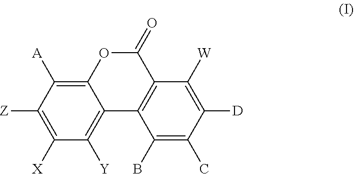

wherein:
A, B, C, D, W, X, Y and Z are each independently selected from H and OH, for example urolitihin A) for increasing fatty acid oxidation and/or decreasing carbohydrate oxidation in immune cells.

The invention further provides a compound of formula (I), or a salt, prodrug, metabolite or derivative thereof;

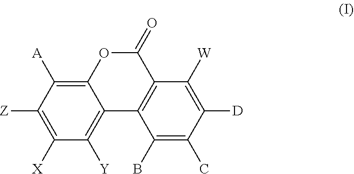

wherein:
   A, B, C, D, W, X, Y and Z are each independently selected from H and OH, for example urolitihin A) for increasing T-cell health and/or function which comprises enhancing one or more of the following pathways:
   1) T-cell receptor signaling;

### 2) RHO GTPases Activate Formins;

### 3) Fcy Receptor-mediated Phagocytosis in Macrophages and Monocytes;

### 4) Natural Killer Cell Signaling—Erria;

### 5) Nuclear Cytoskeleton Signaling Pathway;

### 6) IL-12 Signaling and Production in Macrophages;

7) Neutrophil degranulation;

### 8) Pulmonary Healing Signaling Pathway—0.110;

### 9) FAK Signaling;

### 10) Actin Cyloskeleton Signaling;

### 11) Chronic Myeloid Leukemia Signaling;

12) RHO GTPase cycle;
13) Myelination Signaling Pathway Ii; and/or

### 14) PKCO Signaling in T Lymphocytes.

The invention further provides a compound of formula (I), or a salt, prodrug, metabolite or derivative thereof;

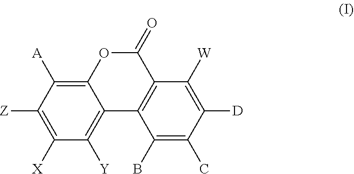

wherein:
   A, B, C, D, W, X, Y and Z are each independently selected from H and OH, for example urolitihin A) for increasing T-cell health and/or function which comprises reducing one or more of the following pathways:
   1) PD-1, PD-L1 pathway
   2) Response to elevated platelet cytosolic Ca2+

### 3) CDX Gastrointestinal Cancer Signaling Pathway

### 4) RAR Activation

### 5) CTLA4 Signaling in Cytotoxic T Lymphocytes

### 6) Protein Kinase A Signaling

### 7) Macrophage Classical Activation Signaling Pathway Z-score

### 8) Coronavirus Pathogenesis Pathway;

9) Role of Osteoblasts in Rheumatoid Arthritis signalling pathway

### 10) Sirtuin Signaling Pathway 2.0;

11) Role of Macrophages, Fibroblast and endothelial cells in rheumatoid arthritis;
12) Cachexia Signaling Pathway; and/or

### 13) Pathogen Induced Cytokine Storm Signalling Pathway.

### Mitochondrial Analysis

The SCENITH technique (described in Argüello et al., SCENITH: A flow cytometry-based method to functionally profile energy metabolism with single cell resolution, 2020, Cell Metabolism, 32, 1063-1075)) enables the measurement of metabolic parameters with single cell resolution and it can be applied to samples of immune cells.

Using the SCENITH technique method, it has been established that Urolithin A treatment potentiates mitochondrial capacity in CD8+ cells. It has been found that Urolithin A treatment causes a shift so that there is less dependence on glucose metabolism and a stronger capacity to shift to OXPHOS to meet metabolic needs when deprived of glycolysis. This is in line with superior mitochondrial function. There is no significant change in the metabolic profiles in the cells from subjects treated with placebo. This confirms that the effects observed upon Urolithin A treatment are due to a boost of mitochondrial function.

### Diagnostic Methods and Kits

The invention further provides a method of establishing whether a drug or other treatment of a human subject is exerting its effects by improving mitochondrial health (for example mitochondrial immune health) in the subject comprising analysing a blood sample (for example using the SCENITH technique) taken before the treatment and comparing it with a blood sample taken after the treatment has taken place. This has use in assessing whether a drug treatment is being effective, or in elucidating the mechanism of action of the drug. In turn, that finding has use in seeing whether supplementation of the treatment with a compound of formula (I) will be beneficial. The invention provides a diagnostic test for making these assessments.

Such a method may be used to assess if a therapy (which may be by a nutraceutical, a medical food or a drug) is working by improving mitochondrial function systemically. It may also be used to enable dosing of the therapy to be adapted to optimise the effectiveness of the treatment. It has not previously been known to monitor the improvement or reversal of a condition in such a simple way-based on a simple blood sample analysis. The knowledge gained from such an assessment can be used to inform whether an intervention or treatment is being effective and should be continued, to inform whether a particular treatment is suitable for the subject in question, and/or to determine a dose that should be used.

The diagnostic method can be carried out by flow cytometry looking at single circulating immune cells (in particular T cells, NK cells or monocytes as described herein). In particular, it has been found that mitochondrial function of particular cell types can be used as a diagnostic marker. CD8+ T cells and NK cells are particularly influential, for example T_{N}, T_{EM}, T_{CM} and T_{EFF} cells, and CD56^{dim}CD16^{bright} NK cells. The effect on T_{N} cells is particularly marked. That is to say that the invention provides a method of assessing whether a drug or other treatment of a human subject is exerting its effects by improving mitochondrial health comprising analysing a blood sample taken from the subject before and after treatment and analysing whether there has been a change in mitochondrial mass (for example as analysed by staining with a cationic fluorophore that accumulates electrophoretically in mitochondria, for example a dye available under the name MitoTracker Green), mitochondrial membrane potential (for example as analysed by staining with a dye that is responsive to the membrane potential for example MitoTracker Red), mitochondrial biogenesis (for example as anaylsed by assessing the level of PGC alpha) by flow cytometry, for example by intracellular flow cytometry. Details of the SCINETH technique can be found in Argüello et al (2020).

The diagnostic method is most reliable when a set of markers are assessed in combination. For example, at least three markers are assessed, for example at least four, for example at least five markers.

The invention further provides a method of assessing whether a drug or other treatment of a human subject is exerting its effects by improving mitochondrial health comprising analysing a blood sample taken from the subject before and after treatment and carrying out flow cytometry to analyse single circulating cells (for example circulating immune cells; for example T cells, NK cells or other monocytes), for example, using SCINETH, and analysing one or more of the following:
(1) change in mitochondrial mass (for example as analysed by staining with a cationic fluorophore that accumulates electrophoretically in mitochondria, for example a dye available under the name MitoTracker Green),
(2) change in mitochondrial membrane potential (for example as analysed by staining with a dye that is responsive to the membrane potential for example a dye available under the name MitoTracker Red), and/or
(3) change in mitochondrial biogenesis (for example as anaylsed by assessing the level of PGC alpha).

The method of the invention may also be used to establish is the subject is in need of a therapy to enhance mitochondrial function. The method may, further be used during post-cancer recovery to monitor the evolution of the immune system, for example after taking a compound or composition of the invention.

The invention further provides a diagnostic test kit that contains reagents for analysis of a panel of biomarkers as set out herein, for example for analysis of the level of at least 3 immune cell populations selected from the group consisting of: naive CD8+ T cells, effector CD8+ T cells, CD28− CD8+ T cells, B cells, CXCR5+ CD4+ T cells, CD161− CD45RA+ T regulator cells, naive CD4+ T cells, CXCR5+ CD8+ T cells, HLADR− CD38+ CD4+ T cells, Thl7 CXCR5− CD4+ T cells, T cells, CD85j+ CD8+ T cells, CD57+ CD8+ T cells, Th2 non-TFH CD4+ T cells, PD1+ CD8+ T cells, effector memory CD4+ T cells, CD27+ CD8+ T cells, lymphocytes, central memory CD4+ T cells, natural killer (NK) cells, monocytes, Thl TFH CD4+ T cells, CD8+ T cells, CXCR3− CCR6− CXCR5+ CD8+ T cells, Th2 TFH CD4+ T cells, plasmablasts, and CD94+NK cells. The profile obtained by analysis of a sample from a subject can provide a prediction of the subject's response to treatments targeted to immune aging or immune rejuvenation.

In a further embodiment, there is provided a method of analysing a drug, dietary supplement, medical food or other treatment of a human subject to establish whether the treatment is exerting its effects by improving mitochondrial health (for example mitochondrial immune health), to establish whether the agent is being efficacious, or to establish an optimised dose of the treatment comprising:
analysing a blood sample taken before the treatment and comparing it with a blood sample taken after the treatment has taken place; and
carrying out flow cytometry to analyse single circulating cells (for example circulating immune; for example, T cells, NK cells or other monocytes);
analysing whether there has been a change in mitochondrial mass, mitochondrial membrane potential or mitochondrial biogenesis.

In a further embodiment, there is provided a method of measuring immune health comprising analysing one or more of the following:
(1) change in mitochondrial mass (for example as analysed by staining with a cationic fluorophore that accumulates electrophoretically in mitochondria, for example a dye available under the name MitoTracker Green),
(2) change in mitochondrial membrane potential (for example as analysed by staining with a dye that is responsive to the membrane potential for example a dye available under the name MitoTracker Red), and/or
(3) change in mitochondrial biogenesis (for example as anaylsed by assessing the level of PGC alpha).

Such analysis can be conducted in peripheral blood mononuclear cells, or in specific immune cell populations, for example, naïve T-cells, CD8+ T-cells, and or natural killer cells (NK cells).

Mitochondrial health in immune cells, for example, derived from blood, can be used as a surrogate of general health, for example, general health in a human. Therefore, In a further embodiment, there is provided a method of measuring immune health comprising analysing one or more of the following:
(1) change in mitochondrial mass (for example as analysed by staining with a cationic fluorophore that accumulates electrophoretically in mitochondria, for example a dye available under the name MitoTracker Green),
(2) change in mitochondrial membrane potential (for example as analysed by staining with a dye that is responsive to the membrane potential for example a dye available under the name MitoTracker Red), and/or
(3) change in mitochondrial biogenesis (for example as anaylsed by assessing the level of PGC alpha).

Such analysis can be conducted in peripheral blood mononuclear cells, or in specific immune cell populations, for example, naïve T-cells, CD8+ T-cells, and or natural killer cells (NK cells).

As mentioned above, the invention provides use of a compound of formula (I), or a salt, prodrug, metabolite or derivative thereof;

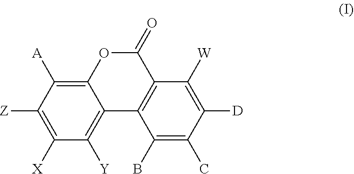

wherein:
A, B, C, D, W, X, Y and Z are each independently selected from H and OH in the manufacture of a medicament for ameliorating immune aging or improving immune health in a human subject.

For example, the treatment, the subject, the compound and other features are as described elsewhere herein.

The invention also provides the use of a compound of formula (I), or a salt, prodrug, metabolite or derivative thereof;

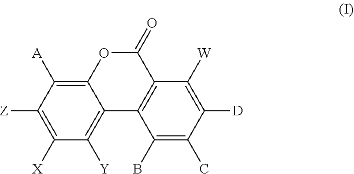

wherein:
A, B, C, D, W, X, Y and Z are each independently selected from H and OH in the manufacture of a medicament for reducing biological age of a human subject, for example as assessed based the degree of DNA methylation in the subject, for example as based on the degree of methylation of DNA in a sample of circulating PBMCs obtained from the subject.

Further, the invention provides a method of ameliorating immune aging or improving immune health in a human subject comprising administering to the subject a compound of formula (I), or a salt, prodrug, metabolite or derivative thereof;

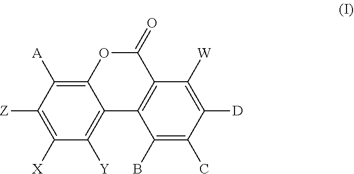

wherein:
A, B, C, D, W, X, Y and Z are each independently selected from H and OH.

For example, the treatment, the subject, the compound and other features are as described elsewhere herein.

The invention further provides a method of reducing biological age of a human subject, for example as assessed based the degree of DNA methylation in the subject, for example as based on the degree of methylation of DNA in a sample of circulating PBMCs obtained from the subject, comprising administering to the subject a compound of formula (I), or a salt, prodrug, metabolite or derivative thereof;

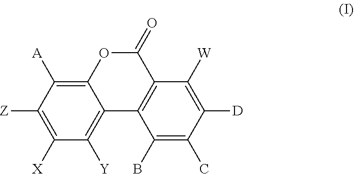

wherein:
A, B, C, D, W, X, Y and Z are each independently selected from H and OH

For example, the biological age is biological immune age or a biological age specific to a target organ, for example skin biological age, brain biological age or muscle biological age.

More specifically, the invention provides a method of increasing the circulating lymphocyte count in a human subject comprising administering to the subject a compound of formula (I), or a salt, prodrug, metabolite or derivative thereof.

The invention also provides a method of increasing the ratio of CD8+ (T_{N}) T cells to CD8+(T_{EM}) T cells in a human subject so that the proportion of naïve CD8+ T cells is increased, comprising administering to the subject a compound of formula (I), or a salt, prodrug, metabolite or derivative thereof.

The invention also provides a method of increasing the proportion of CD56^{dim}CD16^{bright} NK (Natural Killer) cells in the total PBMC population in a human subject, comprising administering to the subject a compound of formula (I), or a salt, prodrug, metabolite or derivative thereof.

The invention also provides a method of increasing mitochondrial function in immune cells in a human subject, comprising administering to the subject a compound of formula (I), or a salt, prodrug, metabolite or derivative thereof. For example, wherein the immune cells ae selected from T-cells (for example, Naïve T cells (T_{N}-cells), T cell memory cells (T_{CM} cells), T effector memory cells (T_{EM}) and or natural killer cells (NK cells).

The invention also provides a method of increasing mitochondrial numbers or mitochondrial mass in immune cells in a human subject, comprising administering to the subject a compound of formula (I), or a salt, prodrug, metabolite or derivative thereof. For example, wherein the immune cells ae selected from T-cells (for example, Naïve T cells (T_{N}-cells), T cell memory cells (T_{CM} cells), T effector memory cells (T_{EM}) and or natural killer cells (NK cells).

The invention also provides a method of increasing mitochondrial biogenesis in immune cells in a human subject, comprising administering to the subject a compound of formula (I), or a salt, prodrug, metabolite or derivative thereof. For example, wherein the immune cells ae selected from T-cells (for example, Naïve T cells (T_{N}-cells), T cell memory cells (T_{CM} cells), T effector memory cells (T_{EM}) and or natural killer cells (NK cells).

The invention also provides a method of decreasing in inflammatory markers in a human subject, comprising administering to the subject a compound of formula (I), or a salt, prodrug, metabolite or derivative thereof.

### Compounds of Formula (I): Urolithins

Compounds of formula (I) (Urolithins) are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

| | |
|---|---|
| | (I) |
| 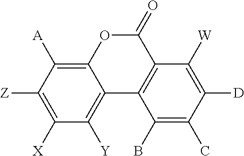 | |

| | Substituent of structure (I) | | | | | |
|---|---|---|---|---|---|---|
| | | | | | W, X | |
| | A | B | C | D | and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (I) may be used in the invention.

In one aspect of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the present invention is urolithin A, urolithin B, urolithin C or urolithin D. Most preferably, the urolithin used is urolithin A.

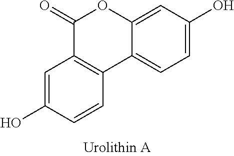 [SMILES: O=c1oc2cc(O)ccc2c2ccc(O)cc12]

According to one embodiment there is provided a compound for use, a composition, a use or a method of the invention wherein the compound of formula (I) is urolithin A.

According to one embodiment there is provided a compound for use, a composition, a use or a method of the invention wherein the compound of formula (I) is urolithin B.

According to one embodiment there is provided a compound for use, a composition, a use or a method of the invention wherein the compound of formula (I) is urolithin C.

According to one embodiment there is provided a compound for use, a composition, a use or a method of the invention wherein the compound of formula (I) is urolithin D.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

The urolithin used in compositions of the invention is preferably a high purity urolithin, for example a high purity urolithin A. For example, it the urolithin in the composition has a purity of over 95% w/w, for example >97% w/w, for example >98% w/w, for example >98.5% w/w; for example >99% w/w; for example >99.5% w/w. The composition thus contains less than 5% w/w of urolithin by-products or synthetic/preparative intermediates; for example the composition contains <3% w/w of urolithin by-products or synthetic/preparative intermediate, for example <2% w/w, for example <1% w/w of urolithin by-products or synthetic/preparative intermediates.

The purity of the urolithin is generally assessed by HPLC assay. In the assay, the urolithin in the composition may, for example have a purity of over 95% area under the curve, for example >97% AUC, for example >98% AUC, for example >98.5% AUC; for example >99% AUC; for example >99.5% AUC.

In some preferred embodiments, the use or method comprises administration of the compound of formula (I) or salt thereof (e.g. urolithin A), in micronized form. Micronization enables the compound of formula (I) to disperse or dissolve more rapidly. Micronisation can be achieved by methods established in the art, for example compressive force milling, hammer milling, universal or pin milling, or jet milling (for example spiral jet milling or fluidised-bed jet milling) may be used. Jet milling is especially suitable. If micronized compound is used, then preferably the compound has a D₅₀ size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 4.0 to 10 μm, for example 1.5 to 7.5 μm, for example 2.8 to 5.5 μm. Preferably, the compound has a D₉₀ size of under 100 μm. More preferably, the compound has a D₉₀ size of under 75 μm, for example under 50 μm, for example under 25 μm, for example under 20 μm, for example under 15 μm. The compound preferably has a D₉₀ in the range 5 to 100 μm, for example 5 to 50 μm, for example 8 to 25 μm, for example 5 to 20 μm, for example 7.5 to 15 μm, for example 8.2 to 16.0 μm. Preferably, the compound has a D₁₀ in the range 0.5 to 2.5 μm, for example 0.5 to 2.0 μm, for example 0.5-1.0 μm. Preferably, the compound of formula (I) or salt thereof (e.g. urolithin A) has a D₉₀ in the range 8.2 to 16.0 μm, a D₅₀ in the range 2.8 to 5.5 μm and a D₁₀ in the range 0.5 to 1.0 μm. For example, the compound of formula (I) or salt thereof (e.g. urolithin A) has a D₉₀ in the range 5.2 to 24 μm (for example 11.9 μm), a D₅₀ in the range 2.0 to 8 μm (for example 3.3 μm) and a D₁₀ in the range 0.1 to 2.5 μm (for example 0.9 μm). For example, the compound of formula (I) or salt thereof (e.g. urolithin A) has a Do in the range 11.6 to 23 μm (for example 16 μm), a D₅₀ in the range 4.9 to 9.2 μm (for example 6.4 μm) and a D₁₀ in the range 1.3 to 2.5 μm (for example 1.9 μm).

In a further embodiment, the compound of formula (I) or salt thereof has a size distribution selected from one of the following:
(i) D₅₀ size in the range 0.5 to 50 μm and a Doo size in the range 5 to 100 μm,
(ii) D₉₀ size in the range 8.2 to 16.0 μm, a D₅₀ size in the range 2.8 to 5.5 μm and a D₁₀ size in the range 0.5 to 1.0 μm;
(iii) Do size in the range 8 to 25 μm, a D₅₀ size in the range 4 to 10 μm and a D₁₀ size in the range 0.5 to 2.0 μm;
(iv) D₅₀ size in the range 0.5 to 20 μm and a Doo size in the range 5 to 50 μm;
(v) D₅₀ size under 50 μm and a D₉₀ size under 75 μm;
(vi) D₅₀ size under 25 μm and a D₉₀ size under 50 μm;
(vii) D₅₀ size under 10 μm and a D₉₀ size under 20 μm;
(viii) D₅₀ size under 10 μm and a D₉₀ size under 15 μm; or
(ix) D₅₀ size of 10 μm and a D₉₀ size of 20 μm.

### Metabolites of Urolithin

In the systemic circulation in humans, urolithins occur mainly a glucuronide conjugates following phase II metabolism. For Urolithin A, such glucuronide conjugates include urolithin A 3-glucuronide and A 8-glucuronide. Other metabolites of urolithins include sulphates. For Urolithin A, such sulfate conjugates include urolithin A 3-sulfate and A 8-sulfate.

### Prodrugs and Precursors of Urolithin

As mentioned above, compounds of formula (I) (Urolithins) are metabolites produced by the action of mammalian, including human, gut microbiota on ellagitannins and ellagic acid. These precursor compounds can be considered to be pro-drugs of the compounds of formula (I)

### Urolithin Administration/Dosage Regimes

In the use of the invention, 200 mg to 2500 mg of the compound of formula (I) is typically administered. As described in further detail below, a preferred compound of formula (I) is Urolithin A. That is to say that 200 mg to 2500 mg of Urolithin A is typically administered.

The administration of compositions of the present invention preferably involves oral administration of a urolithin of formula (I) or salt thereof to a subject in a daily amount in the range of 1.7 to 6.0 mmol per day, for example, from about 1.7 to about 2.7 mmol per day, or from about 2.8 to about 6.0 mmol per day. As discussed below, administration is preferred in the range 125 mg to 2000 mg urolithin A (which corresponds to about 0.55 to 8.8 mmol), 250 mg to 2000 mg urolithin A (which corresponds to about 1.1 to 8.8 mmol), for example 250 mg to 1500 mg, such as 250 mg to 1000 mg, which results in a surprisingly good pharmacokinetic profile. In one embodiment the dose is 125 mg/day, in an alternative embodiment the dose is 250 mg/day, in an alternative embodiment the dose is 500 mg/day and in another embodiment the dose is 1000 mg/day. In a further embodiment, the dose is 1500 mg/day. In a further embodiment, the dose is 2000 mg/day.

In a further embodiment, administration doses are selected from:
250 mg once or twice a day;
500 mg once or twice a day;
750 mg once or twice a day;
1000 mg once or twice a day;
1250 mg once or twice a day; or
1500 mg once or twice a day.

The uses and methods of the present invention involve daily administration of the compound of formula (I) or salt thereof, or of a composition comprising the compound or salt. In some embodiments, the compound or composition is administered once per day, i.e. the compound or composition is to be administered at least once per 24 hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

In some embodiments, the method comprises administering the compound or salt daily for a period in the range of from 7 days to 6 months, from 14 days to 6 months, from 14 days to 5 months, from 7 days to 4 months, from 7 days to 3 months, from 7 days to 2 months, from 7 days to 1 month.

For example, the method comprises administering Urolithin A at a dose of 1000 mg per day for 28 days.

The uses or methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In some preferred embodiments the uses or method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from 400 to 600 mg/day or 400 to 600 mg twice per day. In a preferred embodiment the method involves administration of urolithin A in an amount in the range of from 450 to 550 mg, more preferably approximately 500 mg per day or twice per day.

In other embodiments the methods involve administration of urolithin A in an amount in the range of from 50 to 1000 mg per day or twice per day, or in the range of from 50 to 750 mg, or in the range of from 100 to 500 mg, or in the range of from 150 to 350 mg, for example 250 mg per day or twice per day.

In other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the uses or methods involve administering urolithin A to the subject in an amount in the range of from 4.5 to 11 mg/kg/day, such as 4.5 to 8.5 mg/kg/day. In another embodiment, the uses or methods involve administering urolithin A to the subject in an amount in the range of 5 to 9 mg/kg/day. In another embodiment, the uses or methods involve administering urolithin A to the subject in an amount in the range of from 6.0 to 8 mg/kg/day.

In other preferred embodiments, the uses or methods involve administering urolithin A to the subject in an amount in the range of from 1.8 to 7.1 mg/kg/day, such as 2.5 to 6.5 mg/kg/day. In another embodiment, the uses or methods involve administering urolithin A to the subject in an amount in the range of 3 to 7 mg/kg/day. In another embodiment, the uses or methods involve administering urolithin A to the subject in an amount in the range of from 3.0 to 5.0 mg/kg/day.

In other preferred embodiments, the uses or methods involve administering urolithin A to the subject in an amount in the range of from 9 to 18 mg/kg/day such as 9 to 17 mg/kg/day. In another embodiment, the uses or methods involve administering urolithin A to the subject in an amount in the range of from 10 to 17 mg/kg/day. In another embodiment, the uses or methods involve administering urolithin A to the subject in an amount in the range of from 11 to 16 mg/kg/day.

The compound of formula (I) or salt thereof, or composition containing the compound of salt, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

In a further embodiment, there is provided a composition wherein the compound of formula (I) is administered at a dose of about 1.7-28.6 mg/kg, for example about 1.7 to 15 mg/kg. for example about 3.5-14.0 mg/kg, for example 5.0-10.0 mg/kg.

In a further embodiment for a use of the invention, the compound of formula (I), or a salt, prodrug, metabolite or derivative thereof, for example urolithin A, is administered a dose in the range of about 500 mg and about 1500 mg daily, for example, as a single dose, for example, in the range of about 500 mg to about 1200 mg, for example, in the range 700 mg to about 1200 mg, for example, in the range about 800 mg to about 1200 mg, for example, in the range about 900 mg to 1100 mg, for example, about 500 mg, about 600 mg, about 700 mg, about 800 mg, about 900 mg, about 1000 mg, about 1100 mg about 1200 mg, about 1300 mg, about 1400 mg or about 1500 mg.

In a further embodiment for a use of the invention, the compound of formula (I), or a salt, prodrug, metabolite or derivative thereof, for example urolithin A, is orally administered at a dose sufficient to achieve peak plasma levels of a compound of formula (I), and/or metabolites thereof, of 700-1200 ng/ml. In one embodiment peak plasma levels are between 750-1150 ng/ml of total compound of formula (I), for example, between 800-1100 ng/ml, such as between 800-1000 ng/ml.

In a further embodiment for a use of the invention, the compound of formula (I), or a salt, prodrug, metabolite or derivative thereof, for example urolithin A, is orally administered a dose sufficient to achieve steady state plasma levels of a compound of formula (I), and/or metabolites thereof, of 220-900 ng/ml. In one embodiment, steady state levels are 320-820 ng/ml, for example, 380-730 ng/ml, such as 380-640 ng/ml, such as 450-600 ng/ml, such as about 500 ng/ml.

The ‘steady state’ level is defined as the minimum concentration of total compound in plasma comprising the parent compound, for example, Urolithin A and its metabolites such as urolithin A glucuronide and urolithin A sulphate to which the concentration of total compound falls after 24 hour post dosing, prior to giving the next dose.

In a further embodiment for a use of the invention, the compound of formula (I), or a salt, prodrug, metabolite or derivative thereof, for example urolithin A, is orally administered at a dose sufficient to achieve peak plasma levels of a compound of formula (I), and/or metabolites thereof, of 900-1350 ng/ml. In one embodiment peak plasma levels are between 1000-1250 ng/ml of total compound of formula (I), for example, about 1100 ng/ml, for example about 1000 ng/ml.

In a further embodiment for a use of the invention, the compound of formula (I), or a salt, prodrug, metabolite or derivative thereof, for example urolithin A, is orally administered a dose sufficient to achieve steady state plasma levels of a compound of formula (I), and/or metabolites thereof, of 260-960 ng/ml. In one embodiment, steady state levels are 300-960 ng/ml, for example, 340-940 ng/ml, such as 380-840 ng/ml, such as 380-720 ng/ml, such as about 600 ng/ml. In a further embodiment steady state levels are 400-700 ng/ml.

### Uses

Compounds and compositions of the invention can be used for both therapeutic and non-therapeutic uses, finding use in the treatment of various diseases as well as health conditions not considered to be a disease. Therefore, according to one embodiment of the invention there is provided a compound or composition of the invention for use as a medicament for the treatment of a disease disorder or condition. According to a further embodiment of the invention there is provided a non-therapeutic method of use of a compound or composition of the invention.

The compounds or compositions of the invention can be used as or in a dietary supplement or food product. The compounds or compositions of the invention can also be used as or in a medical food or in a food for medical purposes. A dietary supplement or medical food may contain the compound of the invention in the form of a metabolite, for example a urolithin glucuronide or a urolithin sulfate.

### Therapeutic Uses

Compounds and compositions of the invention find utility in the treatment of disease, disorders and conditions. Therefore, according to a further aspect of the invention, there is provided a compound or composition of the invention for use as a medicament for the treatment of a disease disorder or condition. In particular, the invention is beneficial in the treatment of a disease and non-disease health condition that may be characterised by an aged immune phenotype.

The compounds and compositions of the invention are beneficial for treatments that rely on immune cell populations that generally decline with aging, in particular naïve CD8+ T cells or NK cells. The compounds and compositions of the invention are beneficial for treatments that benefit from improved immune function, for example enhanced phagocytosis.

### Infectious Diseases

According to one embodiment of the invention, there is provided a compound or composition of the invention for use in the prevention or treatment of microbial diseases. In a further embodiment, there is provided a composition of the invention for use in the treatment or prevention of viral diseases.

### Cancer

According to one embodiment of the invention, there is provided a compound or composition of the invention for use in the treatment of cancer. The treatment of many cancers is strongly dependent on the involvement of the subject's immune system. Cancer immunotherapy is a highly effective way of treating certain cancers. However, it can be significantly less effective in a patient with an aged or otherwise compromised immune system. The compounds and compositions of the invention are thus particularly beneficial as a component in a cancer treatment with an anti-cancer treatment, for example an immunotherapy anti-cancer treatment. The compounds and compositions of the invention are also beneficial as a component in the period after a cancer treatment.

### Certain Inflammatory Conditions

According to one embodiment of the invention, there is provided a compound or composition of the invention for use in the treatment of an allergy, asthma or eczema. These are conditions that are driven by IL-4-related inflammation. IL-4 has been shown to be reduced by the compounds or compositions of the invention.

### Additional/Combination Therapy

Compounds and compositions of the invention may comprise one of more further active agents, for use in the treatment or prevention of diseases, disorders or conditions.

Any active agent which is known to be useful, or which has been used or is currently being used for the treatment or prevention of diseases, disorders or conditions can be used with a combination of the invention. See, e.g., Gilman et al, Goodman and Gilman's: The Pharmacological Basis of Therapeutics, 13th ed., McGraw-Hill, New York, 2017; The Merck Manual of Diagnosis and Therapy, Robert S. Porter, M. D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians' Desk Reference (71st ed. 2016) for information regarding therapies (e.g., prophylactic or therapeutic agents) which have been or are currently being used for the treatment or prevention of diseases, disorders or conditions, for example cancer, viral infections or bacterial infections.

For the avoidance of doubt, compounds in compositions of the invention being used in a combination or addition therapy may be formulated in the same composition or formulated in separate compositions for simultaneous, separate or sequential administration.

A compound or composition of the invention can be provided in the form of a supplement or medical food. Such a supplement or food can be taken by a subject alongside a pharmaceutical therapy.

### Non-Therapeutic Uses

Compounds and compositions of the invention find use in method of improving aspects of health in a human subject who is not suffering from any particular condition. For example, in a further embodiment of the invention there is provided the use of a compound or composition of the invention for maintaining or enhancing immune health. That may be especially important at times when the subject is at risk of an infection (eg during a flu season).

In a further embodiment of the invention there is provided the use of a composition of the invention for reducing or slowing inflammaging. Such a use can be considered to decrease the subjects immune biological age.

### Compositions

The uses and methods of the present invention preferably involve oral administration of the compositions of the invention. Any suitable oral composition may be used. Accordingly, the use of a range of compositions which are suitable for oral administration, is envisaged.

Thus in some embodiments, the composition is administered in the form of an oral composition and one or more excipients suitable for oral administration. Oral compositions may comprise compositions having the form of a pill, tablet, capsule, caplet, lozenge, pastille, granules, gummies powder for suspension, oral solution, oral suspension, oral emulsion, syrup, or the like.

In a further embodiment of the invention, the composition is administered by any means known to the skilled person for administration such as, intramuscular, sublingual, cutaneous, inhalation and auricular. Oral administration is preferred.

Compositions may take any physical form suitable for the intended application, for example, they may be in the form of a solid (for example, a tablet or capsule), a semi-solid (for example, a softgel or a gummie), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

As mentioned above, the invention finds use in reducing the biological age of the skin. For such use, a composition is preferably applied topically. A topical formulation of the compound or composition of the invention can be in the form of a cream, an emulsion, an ointment, a lotion or a gel. As well as the active ingredient, such a formulation typically contains inactive ingredients, such as carriers, water, oil, alcohol, propylene glycol, one or more emulsifiers, one or more absorption promoters, one or more fragrances, preservatives and other components. The active ingredient is present at a level that provides sufficient active compound for the formulation to be effective. For example, the active ingredient may be present at a level of 0.1 to 20% by weight, for example 0.2 to 10% by weight, for example 0.25 to 5% by weight, for example 0.5 to 2% by weight, for example 0.5% or 1.0% by weight, for example 1.0% by weight, for example 1.5% by weight, for example 2% by weight.

### Kits

Also within the scope of the present invention are kits, comprising a compound or composition of the invention. Kits typically include a label indicating the intended use of the contents of the kit and instructions for use. The term “label” includes any writing, or recorded material supplied on or with the kit, or which otherwise accompanies the kit.

The invention further provides kits comprising:
(a) a compound or composition of the invention; and
(b) a container, or containers, for containing said compound or composition; and
(c) optionally instructions for simultaneous, separate or sequential administration.

### CAR-T Cells

CAR-T cells (chimeric antigen receptor on T-cells) is a newly developed line of immunotherapy that has shown promise in blood cancer and solid tumours over traditional cancer treatment modalities. In this process (adapted from https://www.cancer.gov/about-cancer/treatment/research/car-t-cells and Wang et al (2023) Cancers 15 (8), 2351 (https://www.mdpi.com/2072-6694/15/8/2351), a patient's own blood cells (PBMC-peripheral blood mononuclear cells) are harvested and T-cells are isolated. CAR-T cells are then created from these isolated T-cells by expressing the candidate cancer/viral antigen using a lentiviral system. These CAR-T cells are then activated and expanded in cell culture and millions of these CAR-T cells are then infused back into the patient's body where they specifically target and kill cancer cells expressing the cancer antigen. These newly created CAR-T-cells are a mix of both CD4 and CD8 T-cells. It is this ratio of CD4: CD8 T-cells that dictates the therapeutic efficacy and toxicity of these CAR-T cells with a higher frequency towards CD8 T-cells driving a better response https://pubmed.ncbi.nlm.nih.gov/37789569/.

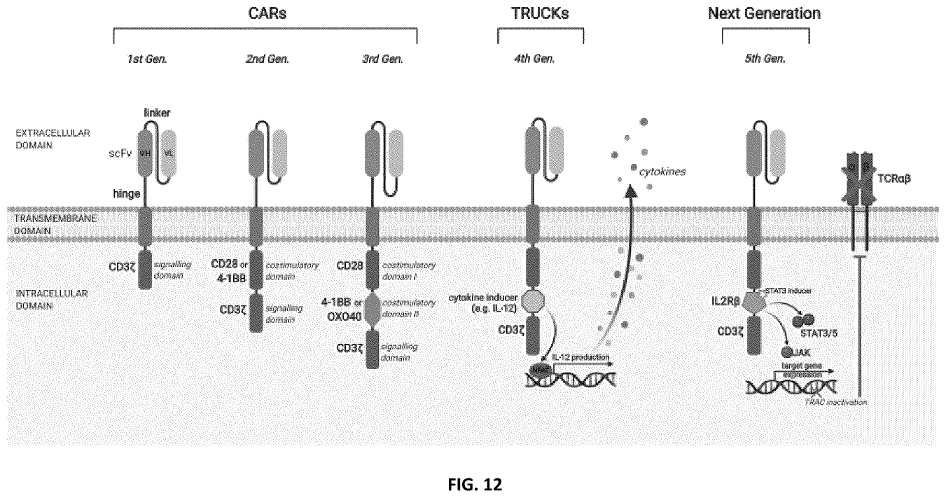

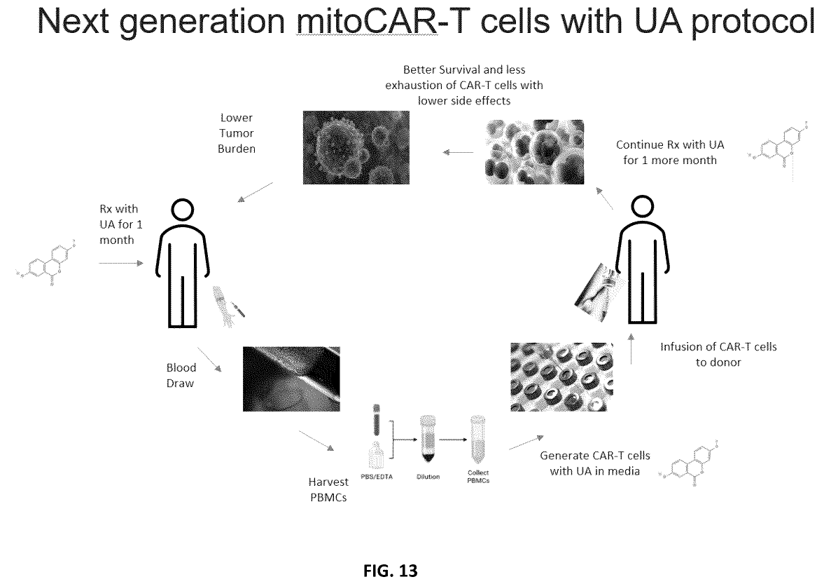

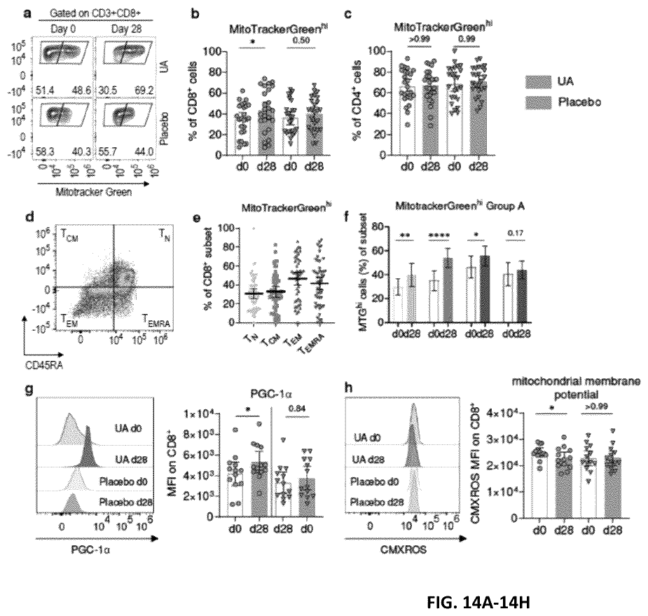

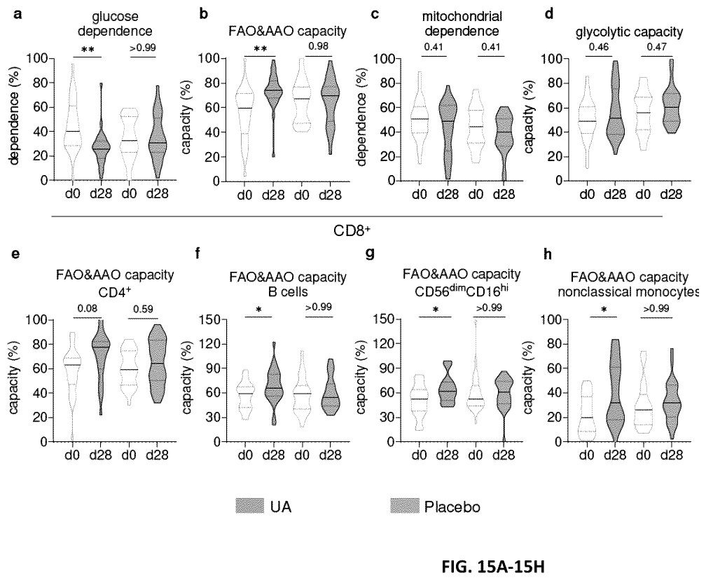

Many generations of CAR-T cells therapies have been improved upon over the years starting from the first generation which simply expressed the T-cell receptor (CD3) to the next ones which added the co-stimulatory molecule (CD28, 41-BB, OX40) to drive a sustained CAR-T cell response following adoptive transfer (see FIG. 12) (see British Journal of Cancer 2019, 120, 26*37). The newer generation CAR-T cells therapies add cytokine inducers and signaling molecules to potentiate the killing activity of these CAR-T cells. However the main problem that persists to this day is the rapid exhaustion and lack of persistence of transfused CAR-T cells. Exhausted CAR-T cells upregulate expression of genes associated with terminated T cell differentiation, aerobic glycolysis and apoptosis (see Oncoimmunology 2021, 10 (1), 1866287). Among cell exhaustion characteristics, impaired mitochondrial function and dynamics are considered hallmarks in these cells.

We have developed, a 6^{th} generation of CAR-T cells with enhanced efficacy and resistance to exhaustion due to superior stemness potential and lower side effect profile (dampening of cytokine storm) by adding a mitochondrial boosting agent in conjunction with the CAR-T cell infusion to a patient (mitoCAR-T cells).

According to a further aspect of the invention, there is provided a method of preparing a CAR-T cell preparation, wherein said method comprises the steps of:
(a) optionally treating a patient with one or more mitophagy inducers (for example, urolithin A), for example, treating a patient for at least 2 weeks, for example, about 1 month with the one or more mitophagy inducers;
(b) obtaining a sample comprising T-cells from the patient, for example, obtaining a blood sample;
(c) isolating T-cells from the sample;
(d) Transfecting the T cells with a CAR (chimeric antigen receptor) gene, to prepare CAR-T cells; and
(e) expanding the number of CAR-T cells, for example, in media containing one or more mitophagy inducers (for example, urolithin A), to prepare a CAR-T cell preparation.

According to a further aspect of the invention, there is provided a method of adoptive cell therapy, wherein said method comprises the steps of:
(f) optionally treating a patient with one or more mitophagy inducers (for example, urolithin A), for example, treating a patient for at least 2 weeks, for example, about 1 month with the one or more mitophagy inducers;
(g) obtaining a sample comprising T-cells from the patient, for example, obtaining a blood sample;
(h) isolating T-cells from the sample;
(i) Transfecting the T cells with a CAR (chimeric antigen receptor) gene, to prepare CAR-T cells;
(j) expanding the number of CAR-T cells, for example, in media containing one or more mitophagy inducers (for example, urolithin A), to prepare a CAR-T cell preparation;
(k) Administering the CAR-T cell preparation to the patient or a different patient; and
(l) treating the patient with one or more mitophagy inducers (for example, urolithin A), for example, treating the patient for at least 2 weeks, for example, about 1 month with the one or more mitophagy inducers.

In a further embodiment, there is provided a CAR-T cell preparation, prepared as described above.

In a further embodiment, there is provided a CAR-T cell preparation, prepared as described above, for use in the treatment of disease, for example, for use in the treatment of cancer.

In a further embodiment, there is provided a CAR-T cell preparation, prepared as described above, for use in the manufacture of a medicament for the treatment of disease, for example, for use in the treatment of cancer.

In a further embodiment, there is provided a CAR-T cell preparation, prepared as described above, for use in the treatment of disease, for example, for use in adoptive cell therapy.

In a further embodiment, a further linker may be added to the chimeric antigen receptor linker, for example selected from: CD3, CD28, 4-1BB, OXO40, a cytokine inducer (for example Il-12), Il-2 and/or an IL2 receptor, for example, IL2-Rβ.

In a further embodiment a mitochondrial target potentiating linker can also be added to the CAR-T cell linker.

In one embodiment, the mitophagy inducer is selected from: urolithin A, kinetin triphosphate, pifithrin-a, deferiprone, metformin, 1,10′-phenanthroline, ciclopirox olamine, nicotinamide riboside, nicotinamide, nicotinamide mononucleotide, resveratrol, fisetin, p62/sqstm1-mediated mitophagy inducer (PMI), spermidine, actinonin and rapamycin.

Methods and use of the invention comprise administration of a compound of formula (I), or a salt, prodrug, metabolite or derivative thereof; as defined herein, to a human, for example, a healthy human.

The term ‘about’ refers to a tolerance of ±20% of the relevant value, for example ±15% of the relevant value, such as ±10% of the relevant value or ±5% of the relevant value.

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity or enhancing solubility.

The term ‘immune revival’ refers to the process of strengthening and enhancing the body's natural defense system to improve its ability to fight off infections and diseases.

The term “pharmaceutically acceptable” means approved by a regulatory agency of the Federal or a state government or listed in the U.S. Pharmacopeia or other generally recognized pharmacopeia for use in animals, and more particularly in humans.

The term, “separate” administration means the administration of each of two or more compounds to a patient from non-fixed dose dosage forms simultaneously, substantially concurrently, or sequentially in any order. There may, or may not, be a specified time interval for administration of each the compounds.

The term “sequential” administration means the administration of each of two or more compounds to a subject from non-fixed (separate) dosage forms in separate actions. The administration actions may, or may not, be linked by a specified time interval. For example, administering compounds over a specified time such as once every 14 to 21 days.

The term “simultaneous” administration means the administration of each of two or more compounds to a subject in a single action such as where each compound are administered independently at substantially the same time or separately within time intervals that allow the compounds to show’ a cooperative therapeutic effect.

The term “systemic inflammation” refers to a widespread inflammatory response that affects the entire body, rather than being localized to a specific area.

The term “therapeutically effective amount” as used herein refers to the amount of a compound or compounds that, when administered, is sufficient to assist in the prevention of the development of, or to relieve to some degree, one or more symptoms of a condition. The particular dose of each compound administered according to this invention will of course be determined by the particular conditions surrounding the case, including the compound administered, the route of administration, the particular condition being treated, as well as considerations such as age, weight and sex of the treated subject.

The invention will now be illustrated with respect to the following non-limiting examples.

### EXAMPLES

### Example 1: Clinical Trial—Experimental Model and Subject Details

### Trial Design and Study Schedule

The present study is a randomized, double-blind, placebo-controlled interventional study, including healthy participants. Subjects were randomized to receive either Urolithin A (Mitopure, Amazentis SA) orally at a daily single dose of 1000 mg (n=25) or a corresponding placebo (n=25) for a duration of 28 days.

The study was approved by the local Institutional Review Board of the University of Frankfurt, Germany (Ethikkommission des Fachbereichs Medizin der Goethe-Universität, approval number 2022-745_2). Subjects were recruited at the clinical site (Goethe University Hospital Frankfurt) via paper-based flyers and social media campaigns. Recruitment was initiated in March 2023 and the last subjects completed the last visit in December 2023. A total of 79 potential participants were screened for eligibility of which n=50 met all the inclusion and exclusion criteria and were subsequently randomized. Participants underwent three study visits at baseline, after seven days (d7) and after one month (d28), respectively. Blood was drawn on all study visits and adverse events were recorded.

Participant demographics are displayed in Table 1. All participants (n=50) completed all study visits, but one participant of the placebo group (n=1) discontinued the allocated intervention early. No deviations in the study protocol were made. No new concomitant diets or medications were reported. All 50 participants are included in the final analysis for the primary endpoint of the trial as established by the study protocol. As the aforementioned participant who discontinued the intervention early provided all biological samples to complete trial, their dataset was included in the final analysis before unblinding of the trial. The study was registered before start of recruitment (https://clinicaltrials.gov/study/NCT05735886) and was conducted in accordance with the guidelines of the international council of harmonization for Good Clinical Practice (GCP) and the Declaration of Helsinki. The present trial follows the CONSORT reporting guidelines for randomized clinical trials.

Participants were deemed healthy by a study physician as determined by their medical history, anthropometric measurements, physical examination and vital signs. Randomization was performed based on age, gender and BMI. Therefore, baseline characteristics of participants were similar, as shown in Table 1: participants of the intervention groups displayed comparable age, BMI and weight. There were more female participants (60% female vs. 40% male) and they were equally distributed to both groups.

Vital signs (systolic blood pressure, diastolic blood pressure, heart rate) did not differ between both groups at baseline (Table 1).

TABLE 1
Study participant demographics.

| | Group A | Group B |
|---|---|---|
| Gender, Female (%) | 60 | 60 |
| Gender, Male (%) | 40 | 40 |
| Age (years) | 53.68 ± 5.25 | 53.20 ± 6.10 |
| (mean ± SD) | | |
| Weight (kg) | 74.24 ± 16.42 | 75.82 ± 15.41 |
| (mean ± SD) | | |
| BMI (kg/m²) (<35 kg/m²) | 25.25 ± 3.80 | 25.72 ± 4.32 |
| (mean ± SD) | | |
| Systolic blood pressure (mmHg) | 128.4 ± 12.34 | 133.0 ± 12.98 |
| (mean ± SD) | | |
| Diastolic blood pressure | 72.29 ± 11.20 | 71.42 ± 10.72 |
| (mmHg) | | |
| (mean ± SD) | | |
| Heart rate (bpm) (mean) | 66.17 ± 5.81 | 70.9 ± 6.50 |
| (mean ± SD) | | |

### Inclusion and Exclusion Criteria

All study participants provided written informed consent before undergoing randomization. Participants were generally healthy as assessed by a study physician to exclude participants with underlying chronic conditions that may have an effect on the immune system. Study participants were aged between 45 to 70 years and of average body weight (BMI between 20.0 and 34.99 kg/m²).

Subjects agreed to avoid excessive physical activity for the duration of the trial or change their regular diet. Likewise, participants agreed to refrain from starting other dietary supplements for the duration of the trial. Concomitant use of corticosteroids, antibiotics, any anabolic steroid, creatine, protein supplements, casein or branched-chain amino acids (BCAAs), immune-boosting (Vitamin C, Zinc) or mitochondrial (COQ10, NAD+) supplements within 45 days prior to screening disqualified trial participation. Smoking and excessive alcohol use, as defined by current WHO criteria, constituted exclusion criteria. In addition, other exclusion criteria included chronic conditions that may have metabolic consequences, are thought to promote inflammation, or have an effect on trial product absorption (such as gastrointestinal conditions) as assessed by a study physician.

### Example 2: Method Details

### Product, Dosing and Randomization

The investigational product was labelled according to ICH-GCP guidelines and local regulatory specifications and requirements. Thus, the label inserted on the side of the product bottle included an emergency clinical contact addresses that was available at all times, trial reference code, packaging batch number, the blinded study code, as well as detailed instructions for product intake.

The investigational product was provided as a maximum of 4 softgels, with each softgel containing 250 mg Urolithin A (UA) or placebo. UA or placebo softgels were packaged into bottles, each bottle containing 30 softgels. For the purpose of the study, the participants were therefore expected to take 4 softgels on each day. Each subject received 4 bottles for 28-day supply at the start of the study. The participants were instructed to take four softgel capsules in the morning, before food intake, with a glass of water. If a participant forgot a dose, they were instructed to take the next dose according to the trial schedule. Participants were instructed not to exceed the intake of the four soft-gel capsules per day.

The investigational product was blinded into a study code by an unblinded person at the sponsor site, who was not involved in trial design, data collection, analysis or interpretation of the data. Upon study participant inclusion and informed consent, randomization was performed double-blind at the study site, using the eCRFs (Secutrial) web-based, in-built randomization platform. A static stratified block randomization algorithm was used, randomizing participants according to age, gender and BMI. For this block-based randomization strategy, a block size of four was used. All participants received the allocated intervention.

### Compliance

Compliance was assessed by counting the returned unused test product at the final visit, rendering a total of eight unused soft-gel capsules in the case of complete study product intake. Compliance was calculated within the eCRF, dividing the number of dosage units taken by the number of dosage units expected, followed by multiplication by 100. Whenever unused test products were unavailable due to the participants' inadvertent discarding of the softgel-containing capsules, participants were asked to specify their compliance.

### Adverse Event Reporting

Subjects were instructed to contact the principal investigator (i.e. the lead study physician) as soon as possible if he or she noticed a problem with the investigational product to enable direct assessment of the situation. Otherwise, participants were asked for any new adverse events (AEs) during the regular study visits. All adverse events were documented and classified according to description (system organ class-SOC and term), intensity, frequency and outcome. The principal investigator assessed all AEs and decided on their causality. Intensity of AEs was graded in a five point scale (mild, moderate, severe, life-threatening and death related to AE). All AEs recorded in the present study are displayed in Table 2. No life-threatening adverse events were recorded.

A total of nine adverse events (AEs) were recorded during the study period (Table 2). Among those, four were reported in the UA group, whereas five were reported in the placebo group. Most AEs (n=6) were upper respiratory tract infections, among three (n=3) were SARS-CoV2 infections. One participant in the placebo group (n=1) discontinued intake upon a major depressive episode that was deemed unrelated to the intervention. No other differences in adverse events were reported between the two groups, confirming safety and tolerance of UA supplementation in aged adults without prior conditions.

TABLE 2
Total number of post-emergent AEs according to SOC.
No life-threatening adverse events were recorded.
No differences were observed between the groups.

| | | Group A | Group B |
|---|---|---|---|
| | | n (%) | n (%) |
| System Organ Class | Lower Level | Total | Total |
| (SOC) | Term | n = 25 | n = 25 |
| Infections and Infestations | Covid-19 | 1 (4%) | 2 (8%) |
| Infections and Infestations | Upper respiratory | 1 (4%) | 1 (4%) |
| | infection | | |
| Respiratory, Thoracic and | Rhinorrhoea | 1 (4%) | 0 (0%) |
| Mediastinal Disorders | | | |
| Gastrointestinal Disorders | Diarrhoea | 1 (4%) | 0 (0%) |
| Gastrointestinal Disorders | Weight loss | 0 (0%) | 1 (4%) |
| Psychiatric Disorders | Depression | 0 (0%) | 1 (4%) |

### Data Management

Study data were processed in accordance with the principles of Good Clinical Practice. Data was acquired from source documentation and entered for each individual subject electronic case report form (eCRF) into a validated data management system (Secutrial). Data entry was completed by site personnel into the study database system. Reference ranges were provided to query each laboratory parameter used during the study to identify the out-of-range values. The study blind was broken at the end of the study following blind database review. Emergency unblinding for safety reasons was not required during the conduct of the study. Source documents were reviewed to ensure that all items had been completed and that the data provided was accurate and obtained as specified in the protocol. In particular, for each participant the following was reviewed to confirm that: informed consent was obtained and documented; enrolled participants fulfilled all inclusion criteria and did not meet any exclusion criteria; AE/SAE reporting had been performed as applicable; study visits had been conducted as per protocol and information had been recorded in the appropriate place in the source document; the study product was being stored correctly and an accurate record of its dispensation to the study participants was maintained.

### Laboratory Chemistry/Haematology

During the baseline and follow-up visits, blood was drawn by a physician or physician's assistant to assess blood biochemistry parameters and haematology. Analysis was performed using clinically validated systems at the University Hospital Frankfurt, Germany.

### Peripheral Blood Mononuclear Cell (PBMC) Isolation

Peripheral blood (35 ml) was collected by venous puncture into ethylenediaminetetraacetic acid (EDTA)-buffered collection tubes. Blood was drawn by a physician or physician's assistant after written informed consent at inclusion into the trial. EDTA tubes were then spun at 400 g, 5 min to separate plasma from cellular contents. Plasma was immediately frozen and kept at −80° C. for experiments detailed below. Resulting cells were subsequently diluted in PBMC wash buffer (PBS/2% FCS) and transferred to SepMate tubes (StemCell) layered with 15 ml FicollPaque Plus (Merck) or LymphoPrep (Progen). PBMC isolation was then performed according to manufacturer's recommendations. In short, tubes were first spun for 10 min, 1200 g at room temperature. The resulting PBMC layer was then transferred to a new 50 ml tube, filled with 15 ml PBMC wash buffer and spun again at 300 g for 8 min. Cell pellets were again washed in PBMC wash buffer and centrifuged at 400 g for 5 min. Finally, resulting pellet was reconstituted in 1 ml of PBMC freezing medium (FCS/10% DMSO), rendering a single-cell suspension with final cell concentration between 0.5-10×10⁶ cells/ml. Suspensions were quickly transferred into standard cryogenic vials and cryopreserved in a freezing container (Corning CoolCell® FTS30) at −80° C. overnight. For long term storage, cells were kept in vapor phase liquid nitrogen.

### PBMC Thawing

PBMCs were quickly thawed in a 37° C. water bath, then slowly added dropwise into polystyrene tubes containing 1 ml of pre-warmed PBMC medium (DMEM/2% FCS). Empty cryovials were again flushed with warm PBMC medium to collect all remaining cells. Then, tubes were topped to 10 ml of PBCM medium and centrifuged at 1500 rpm for 5 min. Cells were counted and used for subsequent experiments.

### Flow Cytometry Staining and Data Processing

Spectral flow cytometry to broadly analyse immune composition was performed on PBMCs from 25 UA and 25 placebo individuals from the cohort. Flow cytometry staining was performed in three batches, to minimize batch-related effects.

PBMC were thawed and allowed to recover for 3 hours in uncoated cell culture dishes. All samples for any particular donor series were thawed together. Cells were stained with the panel shown in Table 3. For the intracellular panel, cells were stained after recovery with the surface panel, then fixed and permeabilized using the ThermoFisher Foxp3 fix/perm system, followed by staining for intracellular markers. For the surface-only panel, cells were treated with Bafilomycin A1 (0.5 μM) for 45 minutes, followed by 45 minutes incubation with 5-dodecanoylaminofluorescein di-β-D-galactopyranoside (C₁₂FDG) before harvest and surface staining. All cells were analyzed on a Cytek Aurora cytometer. FCS files were exported into FlowJo (BD) for further analysis.

TABLE 3
Antibodies used to identify human immune populations via spectral flow cytometry.

| | | | | Catalogue |
|---|---|---|---|---|
| Specificity | Fluorochrome | Clone | Vendor | Number |
| BCL6 | APC-H7 (=APC- | K112-91 | BD_Biosciences | 563581 |
| | cy7) | | | |
| Blimp-1 | BV421 | 6D3 | BD_Biosciences | 565276 |
| CD11c | eFluor_450 | 3, 9 | Thermo_Fisher | 48-0116-42 |
| CD123 | Super_Bright_436 | 6H6 | Thermo_Fisher | 62-1239-42 |
| CD127 | APC-R700 | HIL-7R-M21 | BD_Biosciences | 565185 |
| | (=AF700) | | | |
| CD14 | BUV615 | M5E2 | BD_Biosciences | 751150 |
| CD16 | BUV496 | 3G8 | BD_Biosciences | 612944 |
| CD19 | Spark_NIR_685 | HIB19 | BioLegend | 302270 |
| CD27 | PE-Fire_810 | O323 | Biolegend | 302859 |
| CD279 | BUV661 750260 | EH12.1 | | |
| CD3 | PE- | 7D6 | Thermo_Fisher | MHCD0324 |
| | Alexa_Fluor_700 | | | |
| CD4 | cFluor_YG584 | SK3 | CYTEK | R7-20041- |
| | | | | 100T |
| CD45 | PerCP | 2D1 | BioLegend | 368506 |
| CD45RA | BUV395 | 5H9 | BD_Biosciences | 740315 |
| CD56 | BUV737 | NCAM16.2 | BD_Biosciences | 564447 |
| CD8 | BUV805 | SK1 | BD_Biosciences | 612889 |
| Eomes | PE-Cy7 | WD1928 | Thermo_Fisher | 25-4877-42 |
| FOXP3 | PE-Dazzle_594 | 206D | Biolegend | 320126 |
| GATA3 | BV711 | L50-823 | BD_Biosciences | 565449 |
| HLA-DR | BV750 | L243 | Biolegend | 307671 |
| IgD | BV480 | IA6-2 | BD_Biosciences | 566138 |
| IgM | BV570 | MHM-88 | BioLegend | 314517 |
| ki-67 | BV605 | Ki-67 | BioLegend | 350522 |
| P16 | Alexa_Fluor_647 | EPR1473 | Ab cam | ab192054 |
| P21 | APC | | | |
| Pax5 | BV510 | 1H9 | BD_Biosciences | 563191 |
| RORg | BV650 | Q21-559 | BD_Biosciences | 563424 |
| T-Bet | PerCP-Cy5.5 | 4B10 | Biolegend | 644805 |
| TCRγδ | PerCP- | B1.1 | Thermo_Fisher | 46-9959-42 |
| | eFluor_710 | | | |
| Tox | PE | TXRX10 | Thermo_Fisher | 12-6502-82 |
| Viability | LIVE/DEAD_Blue | NA | Thermo_Fisher | L34962 |
| C12FDG/Spidergal | FITC | | Thermofisher | D2893 |
| CCR7 | BV421 | G043H7 | BioLegend | 353208 |
| CD127 | Super_Bright_436 | eBioRDR5 | Thermo_Fisher | 62-1278-42 |
| CD14 | Spark_NIR_685 | | Biolegend | 367150 |
| CD158b | PE | DX27 | Biolegend | 312707 |
| CD159a | AF700 | S19004C | BioLegend | 375119 |
| CD16 | BUV496 | 3G8 | BD_Biosciences | 612944 |
| CD161 | PE- | | Thermo_Fisher | 61-1619-42 |
| | Alexa_Fluor_610 | | | |
| CD19 | BV570 | | Biolegend | 302236 |
| CD25 | PE- | CD25-3G10 | Thermo_Fisher | MHCD2524 |
| | Alexa_Fluor_700 | | | |
| CD27 | APC-H7 | M-T271 | BD_Biosciences | 560222 |
| CD28 | BV650 | CD28.2 | BioLegend | 302945 |
| CD3 | BV510 | SK7 | BioLegend | 344828 |
| CD38 | APC-Fire_810 | HIT2 | BioLegend | 356643 |
| CD4 | cFluor_YG584 | SK3 | CYTEK | R7-20041- |
| | | | | 100T |
| CD45 | PerCP | 2D1 | BioLegend | 368506 |
| CD45RA | BUV395 | 5H9 | BD_Biosciences | 740315 |
| CD56 | BUV737 | NCAM16.2 | BD_Biosciences | 564447 |
| CD57 | eFluor_450 | TB01 | Thermo_Fisher | 48-0577-42 |
| CD8 | BUV805 | SK1 | BD_Biosciences | 612889 |
| CD95 | PE-Cy5 | DX2 | BioLegend | 305610 |
| HLA-DR | BV750 | L243 | BioLegend | 307672 |
| Klrg1 | PE-Fire_810 | SA231A2 | Biolegend | 367733 |
| TCRγδ | BUV615 | 11F2 | BD_Biosciences | 751308 |
| Tigit | BV480 | 741182 | BD_Biosciences | 747843 |
| Viability | LIVE/DEAD_Blue | NA | Thermo_Fisher | L34962 |
| CD138 | PerCP-Cy5.5 | DL-101 | BioLegend | 352309 |
| CD163 | PE-Cy7 | G025H7 | BioLegend | 353720 |
| CD64 | BV711 | vi ma36 | BioLegend | 305041 |
| CD80 | APC | w17149d | BioLegend | 375403 |
| IgD | BV605 | 11-26c.2a | BioLegend | 405727 |
| IgM | BV785 | mhm-88 | BioLegend | 314543 |

Raw data for all 64 output channels were unmixed using library single-color reference controls for each marker. Spectral compensation for the resulting unmixed data was corrected by manually adjusting a 31×31 matrix of spectral overlaps. Once the data was properly compensated, each individual marker was checked for signal and specificity. The resulting data was then manually gated using a representative sample. The gates are then applied to the other samples in the batch, while spot-checking the gating of the other samples for consistency with the first sample gated. Exemplary gating strategies are shown in FIG. 2.

### Mitochondrial Analysis of PBMCs

PBMCs were thawed as depicted above and cultured in T cell medium (RPMI (ThermoFisher Scientific) 10% FBS (South America origin), 10 mM Hepes (Sigma), 1× Non-Essential Amino Acid, 1 mM Sodium Pyruvate, 50 μM β-Mercaptoethanol, 100 U/ml penicillin, 100 μg/ml streptomycin and 2 mM Glutamax (ThermoFisher Scientific)) overnight. After washing, cells were reconstituted in T cell medium containing respective staining agent: for labelling of mitochondrial mass, Mitotracker Green (Invitrogen) was used at a concentration of 40 nM for 30 mins at 37° C. Mitochondrial membrane potential was assessed via MitotrackerRedCMXROS. Cells were then stained with a combination of CD3 vFlour 450, PE-Cy7 CD4, APC-Cy7 CD8, CCR7 APC, CD45RA FITC and Ghost Violet510. To assess expression of peroxisome proliferator-activated receptor gamma coactivator 1-alpha (PGC-1α), cells were rested for four hours after thawing, then stained with the panel above for 15 mins at 4° C. After washing in FACS buffer, cells were permeabilized and fixed using the eBioscience™ Foxp3/Transcription Factor Staining Buffer Set, followed by staining with PGC-1α (sc-518025; D-5; Santa Cruz) antibody. To assess the expression of Parkin, cells were first stained with CD4 BV786, CD8 PECF 594, CD3 BUV 395, CD45RA FITC, CD14 eF450 and live/dead 780, followed by permeabilization and fixation with transcription factor buffer set kit (BD Biosciences). For mitochondrial analysis, cells were finally fixed in 1% PFA and analyzed on a Cytek Aurora cytometer. Samples were unmixed using reference controls generated in combination with stained Ultracomp beads (Fisher Scientific, 01-2222-41).

### Mitochondrial Dependency Analysis of Immune Populations: SCENITH

Single cell energetic metabolism by profiling translation inhibition (SCENITH) was used to determine metabolic dependencies of select immune populations, which was performed as described previously (R. J. Argüello et al. Cell metabolism 2016, 32, 1063-1075). SCENITH reagents kit (inhibitors, puromycin and antibodies) were obtained from www.scenith.com/try-it and used according to the provided protocol. In short, 0.3×106 PBMCs were treated for 15 min with Control (DMSO), 2-Deoxy-Glucose (2-DG; 100 mM), Oligomycin (O; 1 μM) and the combination of 2DG and Oligomycin (DGO) or Harringtonine (H; 2 μg/mL). Following metabolic inhibitors, Puromycin (final concentration 10 μg/mL) was added to cultures for 30 min. After puromycin treatment, cells were washed in PBMC was buffer and stained with the following antibodies for 15 min at 4° C.: PE-Cy7 CD4, APC Cy7 CD8, CD45RA AF700, vFkour 450 CD3, CCR7 APC, BV786 CD14 PercP5.5 CD19, PE-CF594 CD16; CD56 BV711, BV510 Viability dye.

Cells were fixed and permeabilized using the Foxp3 intracellular staining kit (Invitrogen eBioscience) for 1 h. Intracellular staining of puromycin and protein targets was performed for 1 h in diluted (10×) permeabilization buffer at 4° C. Finally, data acquisition was performed using the Cytek Aurora flow cytometer. Samples were unmixed using reference controls generated in combination with stained Ultracomp beads (Fisher Scientific, 01-2222-41) using the SpectroFlo Software. The unmixed FCS files were used for data processing and analysis using FlowJo (BD, version 10.8.1). Immune cell populations of interest were gated manually for downstream analysis. gMFI expression values for Puromycin were exported from FlowJo to calculate metabolic dependencies. The following calculations were used:

$$[ C = gMFI of anti ‐ Puro ‐ Fluorochrome upon Control treatment ] \qquad ( 1 )$$

$$[ 2 DG = gMFI of anti ‐ Puro ‐ Fluorochrome upon 2 DG treatment ] \qquad ( 2 )$$

$$[ O = gMFI of anti ‐ Puro ‐ Fluorochrome upon Oligomycin treatment ] \qquad ( 3 )$$

$$[ DGO = gMFI of anti ‐ Puro ‐ Fluorochrome upon 2 DG + Oligomycin ( DGO ) treatment ] \qquad ( 4 )$$

$$[ Glucose dependence = 100 ( C - 2 DG ) / ( C - DGO ) ] \qquad ( 5 )$$

$$[ Mitochondrial dependence = 100 ( C - O ) / ( C - DGO ) ] \qquad ( 6 )$$

$$[ FAO dependence = 100 ( C - Eto ) / ( C - DGO ) ] \qquad ( 7 )$$

$$[ Glycolytic Capacity = 100 - Mitochondrial dependence ] \qquad ( 8 )$$

$$[ FAAO = 100 - Glucose dependence ] \qquad ( 9 )$$

### T Cell Stimulation Assay

To study the antigen response of T cells, 0.5×10⁶ thawed PBMCs were pre-stained with CellTrace Violet (C34557 ThermoFisher Scientific) according to the manufacturer's instructions. After washing, they were plated in a 24 well plate for 24 h to deplete monocyte populations by plastic adherence. Next, cells in suspension were transferred to a new tube and centrifuged at 350 g for 5 min, followed by reconstitution in fresh T cell medium. Cells were stimulated with human αCD3/C28 stimulation beads (Dynabeads, ThermoFirsher) for four days, using a ratio of 25 μl Beads per 0.5×10⁶ cells. Following the stimulation period, magnetic beads were discarded via magnetic separation and cells were stained with PE-Cy7 CD4, APC-Cy7CD8, CD45RA FITC, CCR7 AF700 and live/dead ef780.

To study cytokine expression, Brefeldin A (Biolegend) was added to stimulated PBMCs for the last 3 hours. Cells were then washed and stained using the panel above, while intracellular staining for cytokine assessment was performed with BD Cytofix/Cytoperm (BD) with 20 min fixation and overnight incubation with αIL-4-CF594, αIFNγ-PE (XMG1.2; 45-7311-82; eBioscience) and αTNFa-APC (MP6-XT22; 563944; BD). Finally, samples were fixed with 1% PFA until data acquisition.

### Phagocytosis Assay

The pHrodo™ E. coli Red BioParticles® Phagocytosis Kit for Flow Cytometry (Invitrogen) was used to assess phagocytotic capacity of monocytes. The particles were inactivated, unopsonized Escherichia coli (K-12 strain) that enables measurement of phagocytic activity based on acidification of particles as they are ingested.

Thawed PBMCs were immediately used for analysis. 25 μL of E. coli Red BioParticles® were added to 0.25×10⁶ PBMCs in 225 μl PBMC medium. As a negative control, 20 μL of PBS were added to 225 μl of PBMCs. Incubation was performed at 37° C. for 45 min, followed by immediate transfer on ice. Cells were then washed in FACS buffer, centrifuged at 1500 rpm for 5 min, followed by another wash step. Cells were stained for 10 min at 4° C. After washing, cells were reconstituted in PBS and subjected to analysis using a Cytek Aurora fow cytometer. Monocytes were identified as CD3−, CD16+ cells.

### Statistical Tests

Unless otherwise indicated, paired t-test for comparisons within a treatment group or two-sided t tests were used for statistical analysis. The number of samples and experiments, as well as statistical test used are reported in each figure legend. All statistical testing were performed at a 0.05 significance level. Tests were considered statistically significant when the calculated p value was <0.05, * p<0.05, ** p<0.01, *** p<0.001 and **** p<0.0001; n.s., not significant.

Error bars correspond to 95% confidence interval and were plotted by GraphPad Prism (version 9). All measurements were taken from distinct samples, no samples were measured repeatedly to generate data. Statistical analysis was confirmed by an independent biostatistician.

### Statistics and Reproducibility

As one of the primary endpoints constitutes changes in mitochondrial function, this trial is powered to detect a difference in mitochondrial activity of immune cells between treatment groups. Sample size estimates were based on statistical simulations by Jacques et al. (FASEB Journal 2020 34, 2978-2986) where a comprehensive literature review was performed to assess range of effect size for 80% power when oxidative function of muscle cells was studied. According to these statistical simulations, 24 participants are considered sufficient for interventions with an expected 11% effect size. Likewise, the sample size chosen (50) is comparable to a similar study in the literature that focused on immune health and mitochondrial function (B. Zhou et al. The Journal of Clinical Investigation 2020, 130, 6064-6063). No samples were excluded for the complete flow cytometry phenotyping constituting the primary endpoint of the present study.

Samples for plasma cytokine analysis were chosen based on the availability of matched samples at baseline visit and after 28 days. Samples were excluded from analysis when the cytokine level was not detectable. Samples from scRNA sequencing were chosen based on availability of remaining samples.

According to the nature of this study, data collection and analysis were performed blind to the group allocation.

### Plasma Cytokine Measurements

Inflammatory cytokines were measured using LEGENDplex™ HU Essential Immune Response Panel (BioLegend) according to the manufacturer's instructions. Plasma samples were diluted 1:1 in assay diluent as recommended and technical duplicates were used for both the standard curve and experimental samples. Analysis was performed with the provided software.

### Example 3: Biochemical Safety Analysis

Previously, long-term Urolithin A (UA) oral administration over 4 months was found to be safe and well tolerated in a cohort of middle-aged adults (A. Singh et al. Cell Reports. Medicine 3, 2022, 100633). We did not find any differences in baseline levels of metabolic markers (Triglycerides, total cholesterol, LDL cholesterol, HDL cholesterol, uric acid), kidney function parameters (serum creatinine) and liver enzymes (AST, ALT, AP, gGT, bilirubin) within the two groups (Table 4). No significant changes were observed after the 28-day intervention period for either the UA or placebo groups.

TABLE 4
Fifty subjects (n = 50) that successfully met all inclusion
and exclusion criteria were randomized to either UA or placebo.
The two study groups displayed similar metabolic, renal and
hepatic parameters. No significant differences were recorded
after the intake period. Statistics were performed within
groups by paired t-testing (n.s., not significant).

| | Group A d 0 | Group A d 28 | p-value |
|---|---|---|---|
| Total Cholesterol | 212.5 ± 33.60 | 213.0 ± 33.48 | 0.89; n.s. |
| (mg/dl) (mean ± SD) | | | |
| LDL cholesterol | 136.6 ± 33.95 | 136.2 ± 30.56 | 0.89; n.s. |
| (mg/dl) (mean ± SD) | | | |
| HDL cholesterol | 66.06 ± 13.51 | 62.98 ± 17.58 | 0.06; n.s. |
| (mg/dl) (mean ± SD) | | | |
| Triglycerides | 154.0 ± 98.53 | 143.2 ± 70.34 | 0.53; n.s. |
| (mg/dl) (mean ± SD) | | | |
| Creatinine | 0.82 ± 0.13 | 0.81 ± 0.11 | 0.80; n.s. |
| (mg/dl) (mean ± SD) | | | |
| Uric acid | 4.86 ± 1.43 | 5.04 ± 1.79 | 0.25; n.s. |
| (mg/dl) (mean ± SD) | | | |
| Total Serum Protein | 7.02 ± 0.38 | 7.06 ± 0.35 | 0.59; n.s. |
| (g/dl) (mean ± SD) | | | |
| Albumin | 4.54 ± 0.24 | 4.55 ± 0.24 | 0.85; n.s. |
| (g/dl) (mean ± SD) | | | |
| ALT | 28.00 ± 24.02 | 29.44 ± 30.27 | 0.38; n.s. |
| (U/I) (mean ± SD) | | | |
| AST | 27.26 ± 11.56 | 28.04 ± 16.10 | 0.76; n.s. |
| (U/I) (mean ± SD) | | | |
| gGT | 24.60 ± 15.90 | 27.13 ± 19.19 | 0.07; n.s. |
| (U/I) (mean ± SD) | | | |
| AP | 75.40 ± 20.60 | 73.28 ± 19.22 | 0.15; n.s. |
| (U/I) (mean ± SD) | | | |
| Bilirubin | 0.53 ± 0.31 | 0.49 ± 0.26 | 0.28; n.s. |
| (mg/dl) (mean ± SD) | | | |
| | Group B d 0 | Group B d 28 | p-value |
| Total Cholesterol | 201.0 ± 34.82 | 201.7 ± 31.40 | 0.87; n.s. |
| (mg/dl) (mean ± SD) | | | |
| LDL cholesterol | 127.3 ± 32.52 | 126.6 ± 27.76 | 0.85; n.s. |
| (mg/dl) (mean ± SD) | | | |
| HDL cholesterol | 62.97 ± 16.85 | 63.93 ± 20.65 | 0.49; n.s. |
| (mg/dl) (mean ± SD) | | | |
| Triglycerides | 114.7 ± 59.62 | 124.1 ± 78.05 | 0.29; n.s. |
| (mg/dl) (mean ± SD) | | | |
| Creatinine | 0.85 ± 0.18 | 0.85 ± 0.20 | 0.83; n.s. |
| (mg/dl) (mean ± SD) | | | |
| Uric acid | 4.76 ± 1.56 | 4.70 ± 1.45 | 0.25; n.s. |
| (mg/dl) (mean ± SD) | | | |
| Total Serum Protein | 6.90 ± 0.38 | 6.89 ± 0.34 | 0.80; n.s. |
| (g/dl) (mean ± SD) | | | |
| Albumin | 4.50 ± 0.18 | 4.44 ± 0.20 | 0.22; n.s. |
| (g/dl) (mean ± SD) | | | |
| ALT | 19.56 ± 8.34 | 19.68 ± 8.47 | 0.86; n.s. |
| (U/I) (mean ± SD) | | | |
| AST | 23.04 ± 4.56 | 23.08 ± 5.28 | 0.96; n.s. |
| (U/I) (mean ± SD) | | | |
| gGT | 17.64 ± 9.63 | 17.48 ± 9.49 | 0.89; n.s. |
| (U/I) (mean ± SD) | | | |
| AP | 74.48 ± 21.28 | 73.64 ± 22.24 | 0.56; n.s. |
| (U/I) (mean ± SD) | | | |
| Bilirubin | 0.46 ± 0.15 | 0.43 ± 0.15 | 0.25; n.s. |
| (mg/dl) (mean ± SD) | | | |

### Example 4: UA Supplementation Alters the Immune Phenotype in Study Subjects

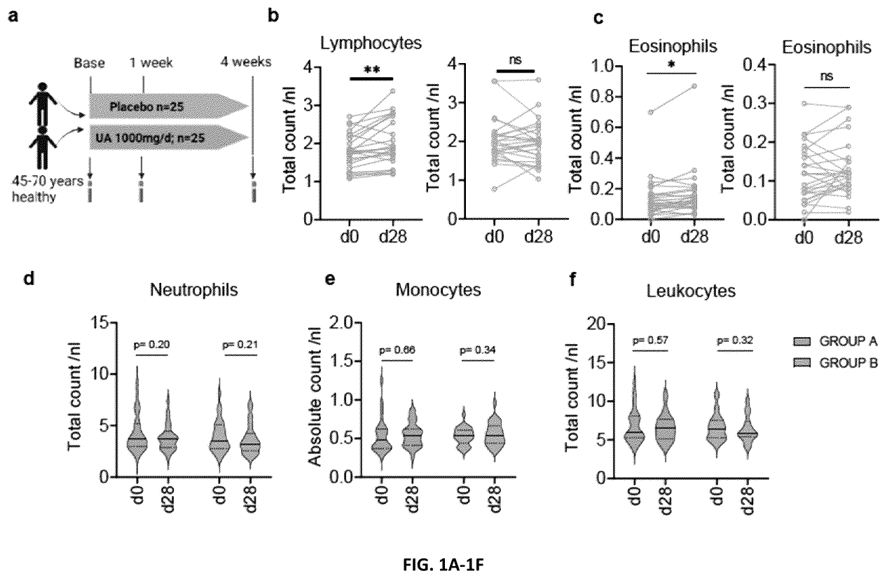

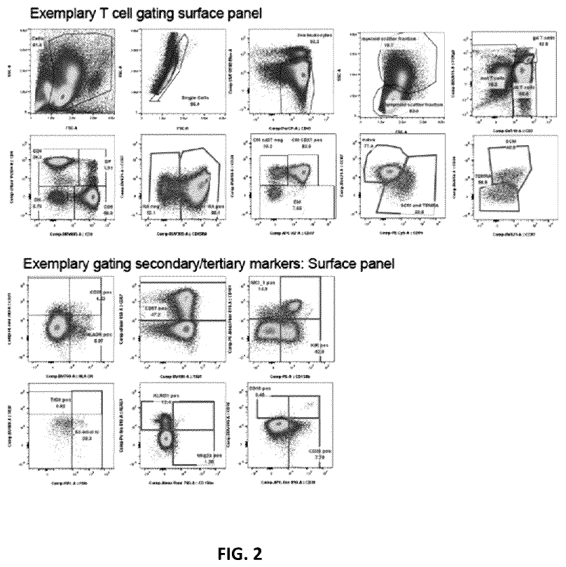

The aim of the present study was to globally assess the effect of UA on the immune system, in particular with respect to parameters of immune aging. We therefore performed clinical hemocytometry to first categorize circulating immune cells, comparing immune composition at baseline and upon the last study visit (FIG. 1A). After the 28 day supplementation period, participants in the UA group displayed significantly more circulating lymphocytes (FIG. 1B) and eosinophiles (FIG. 1C) compared to baseline levels, which was not observed in the placebo group. Other immune populations, such as monocytes, total leukocytes and neutrophiles did not show marked alterations upon any intervention (FIG. 1D-F).

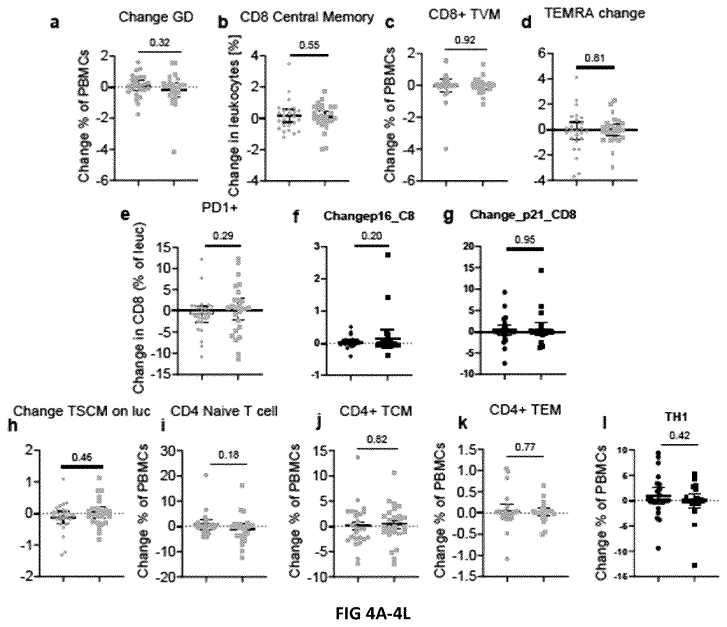

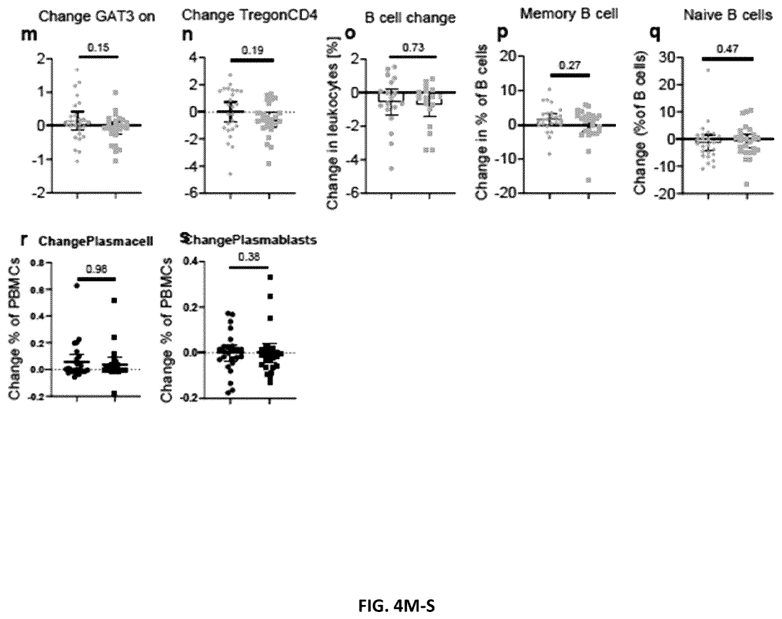

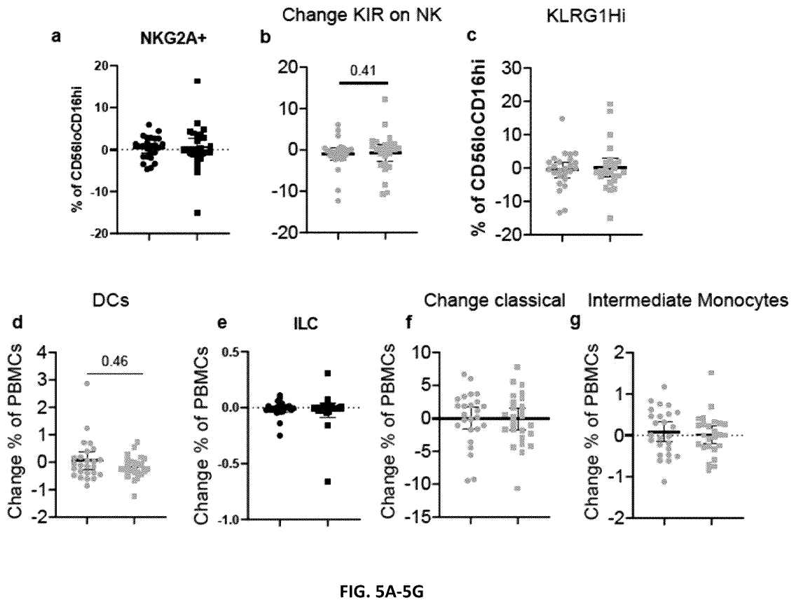

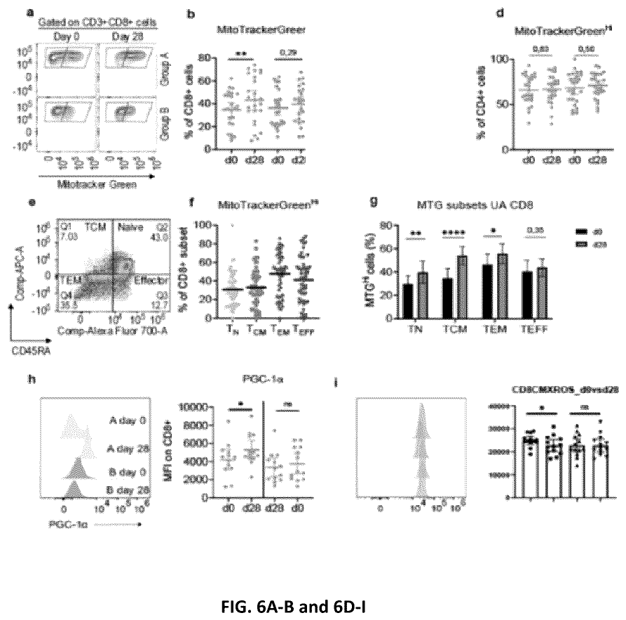

We performed broad spectral cytometry (FIG. 6A), to fully characterize the immune phenotype upon UA intake for the first time. Our panels included >30 markers in a surface marker and transcription factor panel (Table 3; FIG. 2H).

Whereas no differences were found in percentage of total αβCD3+, CD8+, CD4+ T cells or circulating γδ T cells (FIG. 3B-D; FIG. 4A) among total PBMCs analysed upon any intervention, we observed marked changes within CD8+ subpopulations. Contrary to the finding that UA induces TSCM formation in vitro (D. Denk et al. Immunity 2022, 55, 2059-2073), no differences were exhibited between the UA and placebo group (FIG. 3E). Yet, the proportion of naïve T cells (T_{N}) among total PBMCs upon UA intake was increased after UA intake (FIG. 3F), compared to placebo. In accordance to an ameliorated aging phenotype (D. A. Moglikenko et al. Immunology 2013 22, 484-498), effector memory cells (T_{EM}), generally expanded in the elderly (A. Alpert et al. Nature Medicine 2019, 25, 487-495), were concomitantly reduced in UA-treated participants (FIG. 3G). Other CD8+ subsets, such as central memory cells (T_{CM}), TEMRA or recently characterized “virtual memory” cells (TVM) that arise with a memory-like phenotype without prior foreign antigen challenge (B.-C. Chiu et al. Journal of Immunology 2013, 191, 5793-5796), remained unaffected in both cohorts (FIG. 4B-D). However, CD8+ cells in the UA group displayed more Ki67 (FIG. 3H) after the completion of the last study visit, a marker of cellular proliferation and T-cell reinvigoration that predicts pathological complete response to immune checkpoint blockade in patients with triple-negative breast cancer (X. Q. Wang et al. Nature 2023, 621, 868-876). A small reduction was observed in TOX expression (FIG. 3I), the master regulator of T cell exhaustion that marks aging-associated T cells (Taa) and promotes CD8+ T cell dysfunction in cancer (D. A. Mogilenko et al Immunity 2021, 54, 99-115, e12; A. C. Scott Nature 2019, 571, 270-274; X. Wang et al. Journal of Hepatology 2019, 71, 731-741). PD-1 expression was unaltered in both groups (FIG. 4E). Surprisingly, senescence-associated β-galactosidase (SA-β-GAL), a previously identified marker of senescence, was increased in circulating CD8+ cells after UA intake (FIG. 3J). Yet, there was no difference in other prominent senescence markers such as p16 and p21 (FIG. 4F-GH), suggesting that SA-β-GAL expression was indicative of lysosomal number and activity that was elicited by the mitophagy inducer UA. Notably, none of the observed changes were present after placebo (B. Y. Lee et al. Aging Cell 2006, 5, 187-195). Altogether, UA deeply altered the CD8+ phenotype towards a more naïve-like, less exhausted global state.

The CD4+ population also undergoes changes with age that are in part comparable with the ones occurring in CD8+ compartment, displaying a reduction in the T_{N} population with an expansion of T_{EM} and regulatory CD4 cells (T_{reg}9). No changes on CD4+ cells were observed: there was no difference in CD4+ T_{SCM}, T_{N}, T_{EM} or T_{CM} populations (FIG. 4H-K). There were also no changes in CD4+ Th1 cells (marked by T-bet expression), Gata3+ Th2 cells, FoxP3+ T_{reg}. or circulating T follicular helper cells (Tfh1, identified via Bcl-6) (FIG. 4L-N). UA intake therefore did not affect the phenotype of CD4+ cells in the human cohort. There were no changes in percentage of total B cells, plasma cells or plasmablasts among PBMCs or specific B cell subsets between the two intervention groups (FIG. 4O-S).

Apart from pronounced CD8+ T cell changes, some other immune compartments were also affected by UA supplementation. CD56^{dim}CD16^{bright} NK cells, the most common NK subset in the blood, were markedly expanded among total PBMCs in the UA group (FIG. 3J). No change was observed in the expression of their inhibitory receptors such as NKG2A, KIR or KLRG1 (FIG. 5A-C). Circulating DCs and ILCs did not change upon either intervention (FIG. 5D-E). When further interrogating monocyte populations, nonclassical monocytes (defined as CD14^{lo}CD16^{hi} cells) were found to have increased among total PBMCs after the 28 period compared to placebo (FIG. 3L), whereas intermediate monocytes (CD14^{hi}CD16^{hi}) and classical monocytes (CD14^{lo}CD16^{hi}) did not undergo change between the two groups (FIG. 3L, FIG. 5F-G) Yet, classical monocytes exhibited less HLA-DR in participants of the UA group at the final study visit (FIG. 3M), indicative of a less inflammatory phenotype (J. Schulte-Schrepping et al. Cell 2020, 182, 1419-1440, e23).

In conclusion, the data show that UA supplementation instigates profound phenotypical changes in circulating immune cells, with expansion of lymphocyte populations and an emphasis on a naïve-like, less exhausted CD8+ cells.

### Example 5: UA Intake Elicits Mitochondrial Remodelling in Circulating CD8+ Cells

It was assessed whether UA supplementation elicited any mitochondrial remodelling in the immune compartment. MitotrackerGreen (MTG) is a fluorescent dye that stains mitochondria independent of mitochondrial membrane potential. CD8+ cells of UA-treated participants exhibited marked expansion of mitochondrial mass, evidenced by an increased proportion of MTGHi cells after 28 days, but not after seven days, that was absent in the placebo group (FIG. 6A-C). Compared to CD8+ cells, CD4+ cells generally displayed greater mitochondrial mass (FIG. 6D). This was consistent with previous findings in the elderly that support inherent differences in mitochondrial content between CD4+ and CD8+ cells (L. A. Callender et al. Aging Cell 2020, 19, e13067). In accordance to our observation that CD4+ phenotype remained strikingly unaltered after UA intake (FIG. 6C), there was no significant change in mitochondrial content of CD4+ cells after 28 days.

T cell subsets exhibit largely divergent metabolic requirements owed to their functional differences that results in different mitochondrial ultastructures (M. D. Buck et al. Cell 2016, 166, 63-76). Therefore, mitochondrial mass in CD8+ subsets was assessed in detail to examine whether changes observed in mitochondrial content of total CD8+ may represent a by-product of an altered CD8+ distribution. Using the combination of CD45RA and CCR7, CD8+ was divided into T_{N}, T_{EM}, T_{CM} and T_{EFF} subsets (FIG. 6E). Naïve CD8+ exhibited the lowest amount of MTGHi cells, whereas T_{EM} and T_{EFF} retained more mitochondrial mass (FIG. 6F). Increased mitochondrial content was detected in several CD8+ subsets analysed after 28 days of UA supplementation (FIG. 6G). This showed that there had been broad mitochondrial remodelling of the entire CD8+ compartment.

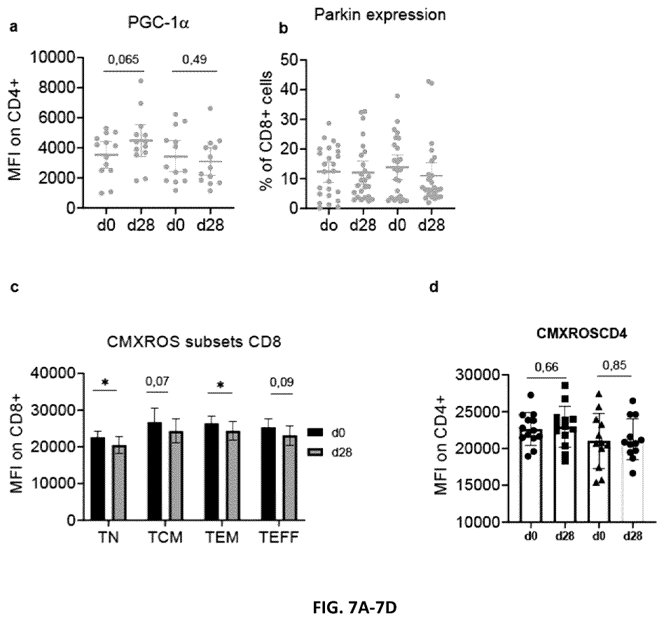

Mitochondrial biogenesis and mitophagy are closely interlinked to guarantee sustained replacement of dysfunctional mitochondria (A. Picca et al. Nature Metabolism 2023, 186, 243-278). Peroxisome proliferator-activated receptor gamma coactivator 1-alpha (PGC-1α) is considered the master regulator of mitochondrial biogenesis (B. N. Finck and D. P. Kelly The Journal of Clinical Investigation 2006, 116, 615-622). It was therefore investigated whether increased mitochondrial content in CD8+ could be due to activation of compensatory mitochondrial biogenesis. CD8+ cells of subjects in the UA group displayed stronger expression of PGC-1α after the intervention period (FIG. 6H), whereas no such change was detected after intake of placebo. Consistent with the lack of mitochondrial expansion, no significant induction of PGC-1α was seen in CD4+ cells (FIG. 7A). Change in Parkin protein expression was detected during the study visits at seven days or 28 days (FIG. 7B-C). This is in contrast to the report of induction of the Pink1/Parkin mitophagy pathway upon UA supplementation in both the human muscle and murine T cells (D. D'Amico et al. Trends in Molecular Medicine 2021, 27, 687-699; D. Denk et al. Immunity 2022, 55, 2059-2073, e8; A. Singh et al. European Journal of Clinical Nutrition 2022, 76, 297-308).

Given that hyperpolarized mitochondria can be a source of reactive oxygen species (ROS) which can potentiate senescence (A. Perl et al. Trends in Immunology 2004, 25, 360-367) and are a characteristic of CD8+ cells in the elderly (S. L. Sanderson and A. K. Simon Aging Cell 2017, 16, 1234-1243), mitochondrial membrane potential (MMP) was assessed. Here, CD8+ cells in the UA group showed significantly lower MMP (FIG. 6I). Low MMP has been postulated to mark T cells with stemness features and decreased oxidative stress (M. Sukumar et al. Cell Metabolism 2016, 23, 63-76). Reduction of MMP was again retained in CD8 subsets and absent in CD4+ cells (FIG. 7D-E).

### Example 6: Metabolic Reprogramming is Induced Upon UA Intake

The immune system exhibits a striking metabolic plasticity, with metabolic reprogramming critically instructing, but also defining immune fate and function (M. D. Buck et al. Cell 2017, 169, 570-586). In T cells, different metabolic needs are met by diverging mitochondrial ultrastructures (M. D. Buck et al. Cell 2016, 166, 63-76).

To further characterize the metabolic profile of immune cells on a single-cell resolution, we employed SCENITH, a flow-cytometry based strategy that uses incorporation of Puromycin to infer metabolic dependencies (see Argüello et al., SCENITH: A flow cytometry-based method to functionally profile energy metabolism with single cell resolution, 2020, Cell Metabolism, 32, 1063-1075).

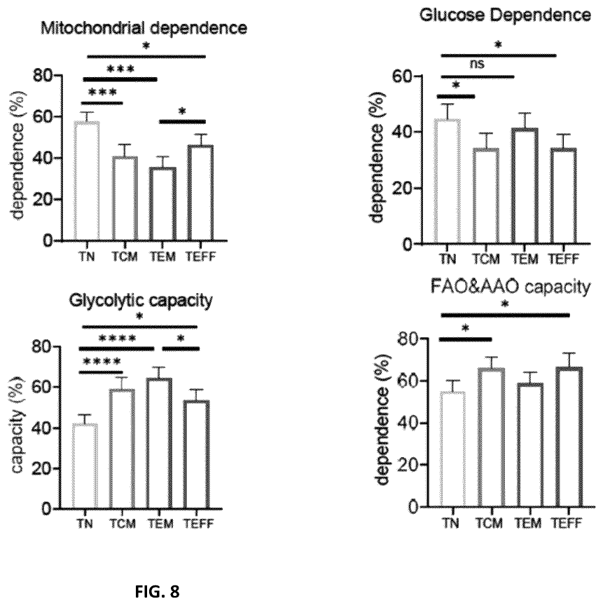

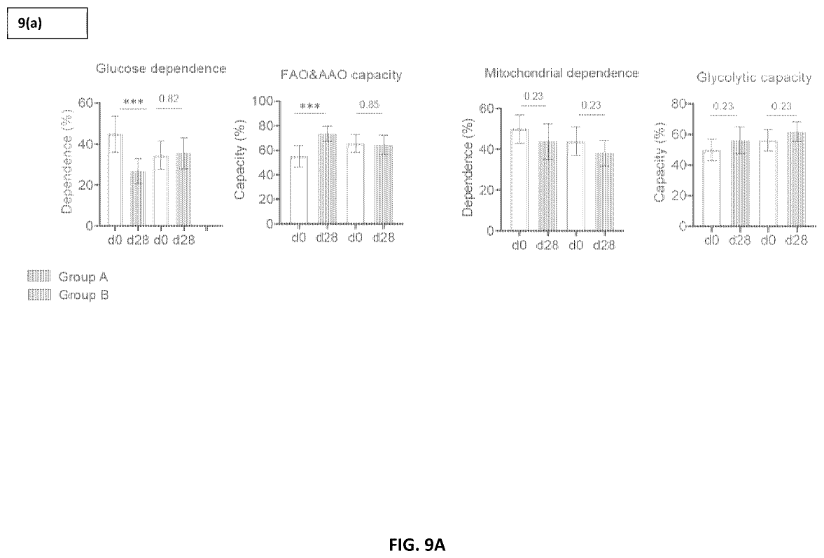

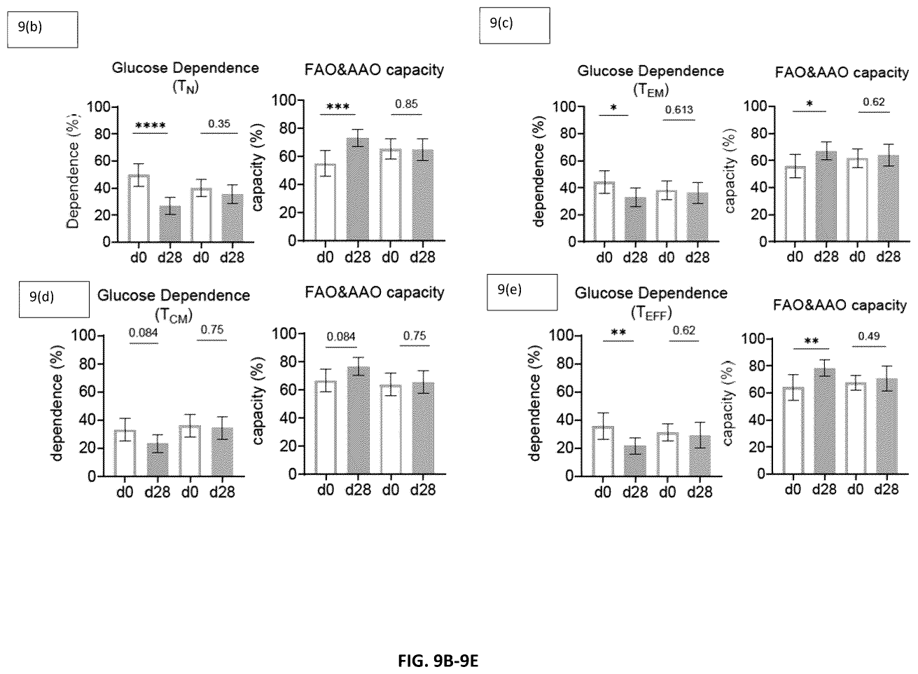

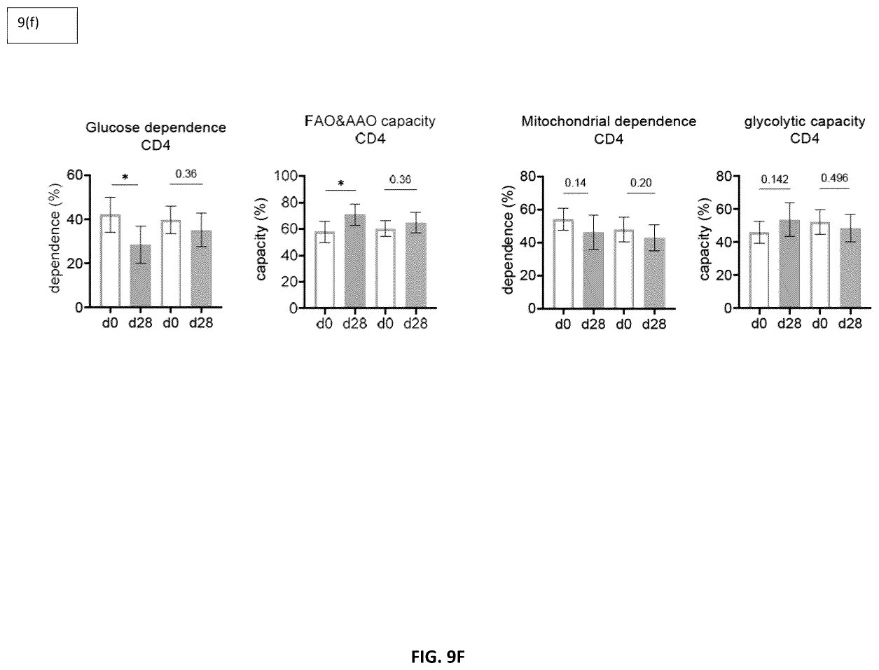

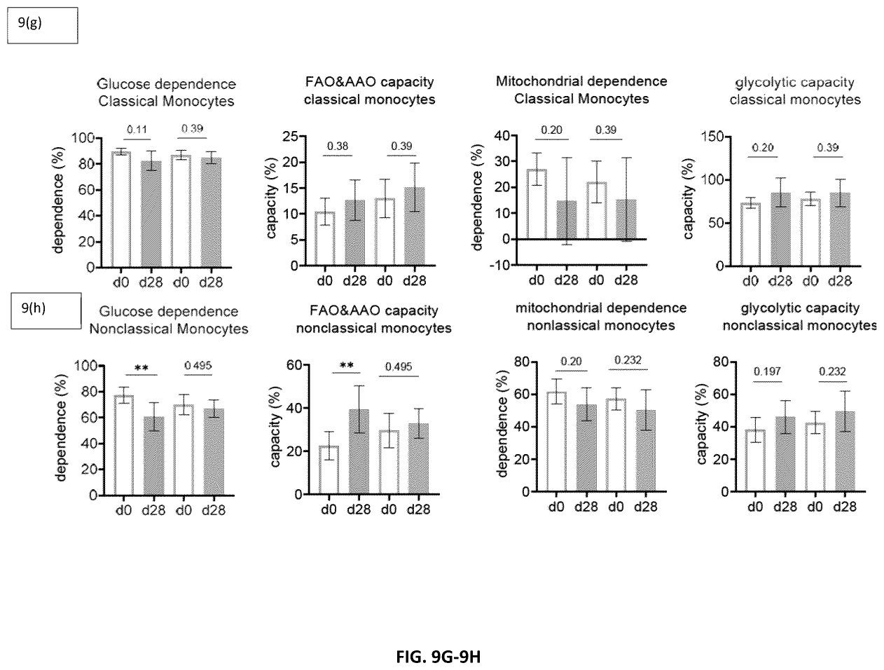

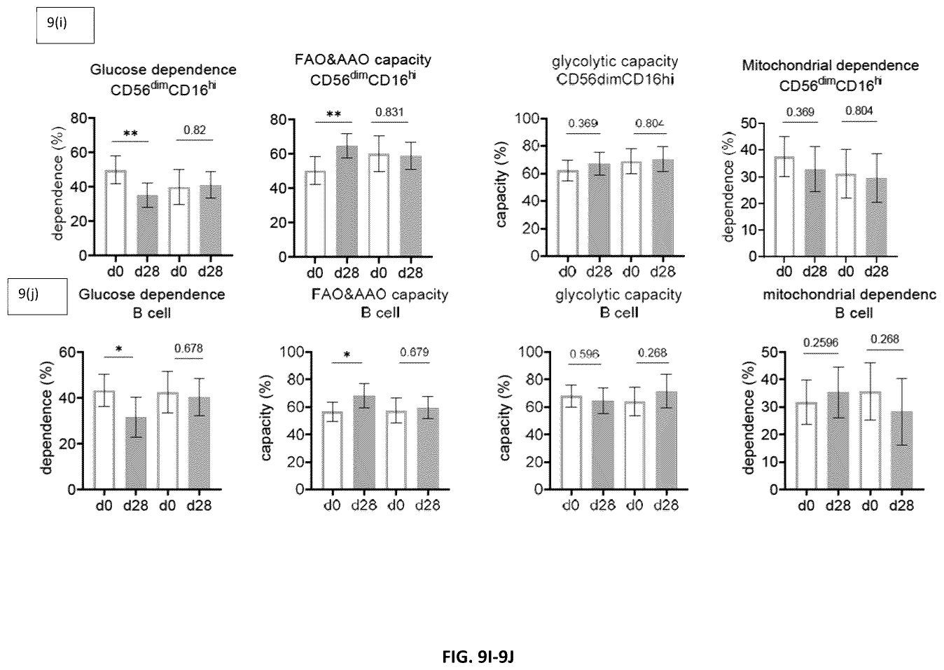

Using the SCENITH technique, subset-specific metabolism is observed. In FIG. 8, the baseline metabolic profile is shown for the CD8+ cells (all participants). Memory subsets rely less on glucose but use fatty acid oxidation. Urolithin A treatment potentiates mitochondrial capacity (FIG. 9). The potentiation is seen in total CD8+ cells (FIG. 9A), and also in each of the subsets: (b) T_{N} cells; (c) T_{EM} cells; (d) T_{CM} cells; and (e) T_{EEF} cells. This means that there is less dependence on glucose metabolism and a stronger capacity to shift to OXPHOS to meet metabolic needs when deprived of glycolysis. This is in line with superior mitochondrial function.

Further cell types were also analysed and the glucose dependence, the FAO&AAO capacity, the glycolytic capacity and the mitochondrial dependence were assessed and the results are shown in FIG. 9F for CD4+ cells; FIG. 9G) for Classical Monocytes; FIG. 9H for Non-classical Monocytes; FIG. 9I for CD56^{dim}CD16^{hi} cells; and FIG. 9J for B cells.

### Example 7: Functional Remodelling of the Immune System Results in Ameliorated Inflammaging

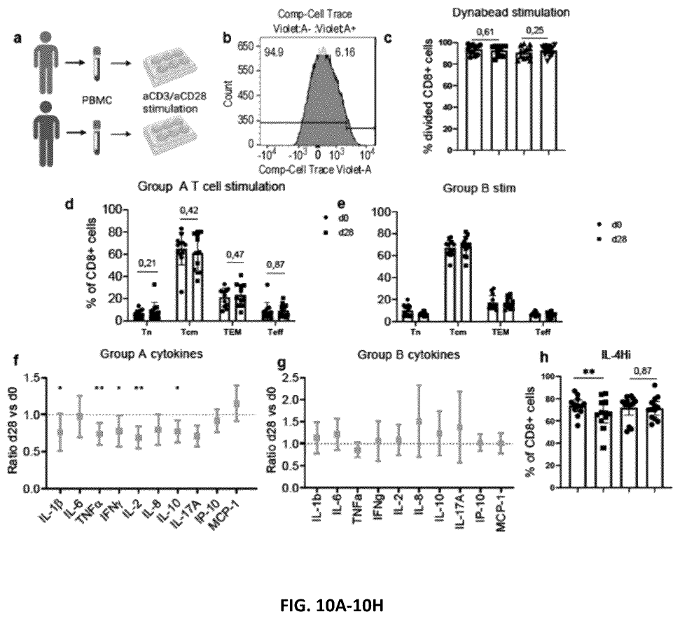

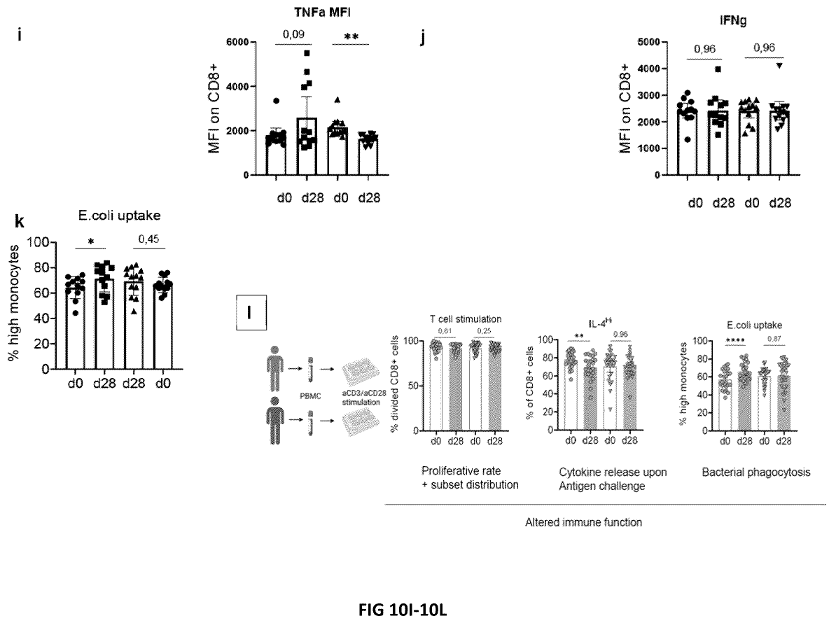

To functionally characterize the human immune system after systemic UA supplementation further, proliferative capacity and cytokine secretion of T cells was assessed. PBMCs from both intervention groups were incubated in the presence of αCD3/αCD28 stimulation beads for a total of four days (FIG. 10A). T cells derived from placebo and UA-treated participants displayed robust proliferation, yet there was no difference between the two interventions or from baseline to the final study visit (FIG. 10B-C). CD8+ cells preferentially took up a T_{CM} and T_{EM} phenotype after CD3 receptor engagement with no treatment-specific distribution pattern (FIG. 10D-E).

The effect of UA supplementation on circulating plasma levels was investigated. Administration of UA led to significantly lower levels of several plasma cytokines, namely TNF-α, IL-1β, IFN-γ, IL-2, and IL-10 (FIG. 10F-G). No difference was found for IL-6, IL-8, IL-17, interferon-gamma induced protein (IP-10) and monocyte chemoattractant protein-1 (MCP-1) after UA or placebo intake (FIG. 10F-G).

We investigated cytokine expression of stimulated T cells to detect potential differences in the context of immune response which could constitute a potential predisposition to conditions that may only manifest when specifically triggered. Antigen challenged CD8+ cells of UA-treated subjects displayed reduced IL-4 production, whereas placebo intake did not elicit any changes (FIG. 10H). Despite the pronounced anti-inflammatory signature observed in the plasma (FIG. 10F), CD8+ expression of IFN-γ or TNF-α remained unchanged when the cells were stimulated (FIG. 10I-J). Taken together, UA treatment resulted in reduced circulating proinflammatory cytokines at steady-state and reduced IL-4 expression in the context of an antigen-provoked T cell response. In FIG. 10L, there is shown a summary of the main effects: the effects on T-cell stimulation, IL-4 production and E. coli uptake.

### Example 8: UA Supplementation Improves Phagocytosis Capability

We investigated the functional capacity of monocytes upon UA intake. For this, a phagocytosis assay to differentially assess monocyte phagocytotic capabilities of gram-negative bacteria was performed. Monocytes from UA-exposed participants exhibited significantly enhanced phagocytosis of E. coli particles ex vivo when compared with the placebo cohort (FIG. 10K), indicating a superior uptake and clearance of gram-negative bacteria.

Collectively, these data show that UA supplementation results in changes in immune function that carries systemic consequences and a clear benefit to immune function.

### Example 9: UA Supplementation Decreases Biological and Immune Age

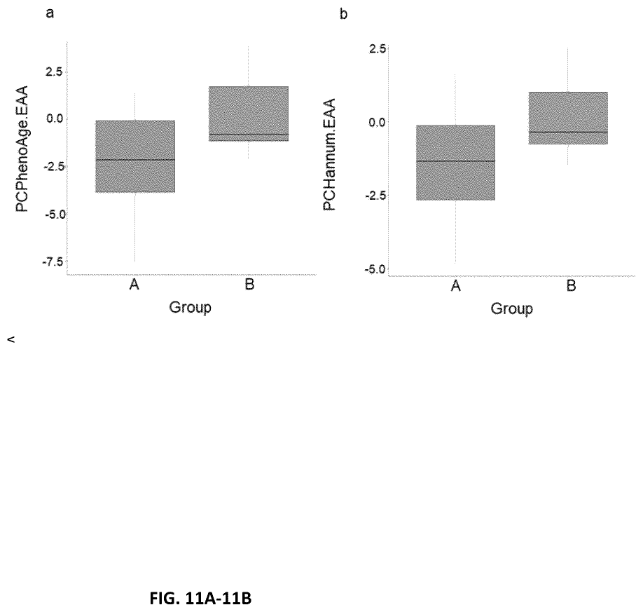

Blood taken from the participants at baseline and the last study visit (d28) time points were applied to dried blood cards and processed by TruDiagnostic™. The test uses DNA methylation data analysis to calculate biological ages using the PhenoAge 2^{nd} generation clock and the Hannum 1^{st} generation clock. The Urolithin A intervention group's biological (immune cell-blood based) age was reduced by a median of about 2 years from the start of the intervention, as calculated by the PhenoAge clock (FIG. 11A). A similar decrease in aging was found in the Hannum clock (FIG. 11B). A decrease in biological age was not observed in the Placebo group.

### Example 10: Single Cell RNA Sequencing Reveals UA-Mediated Transcriptomic Alterations

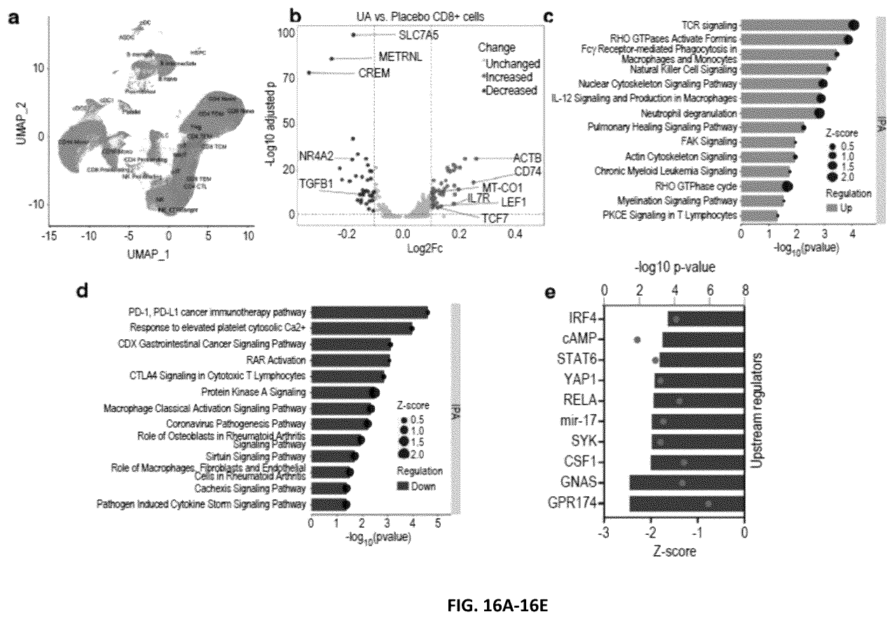

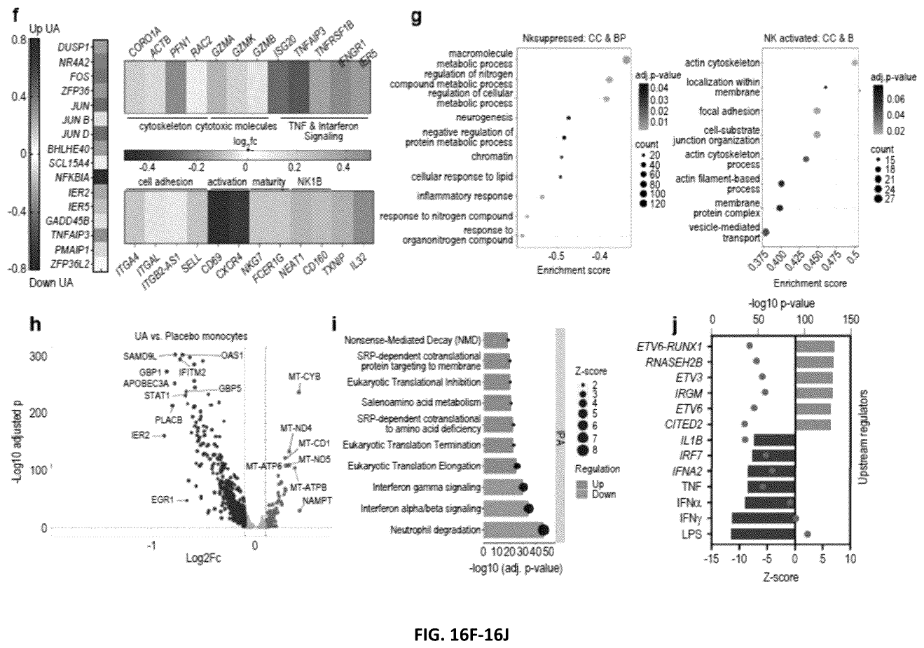

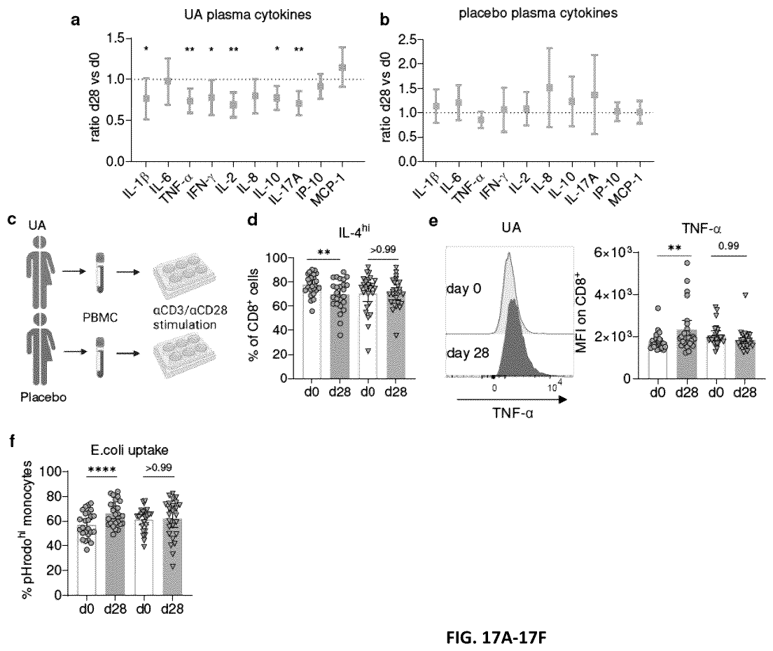

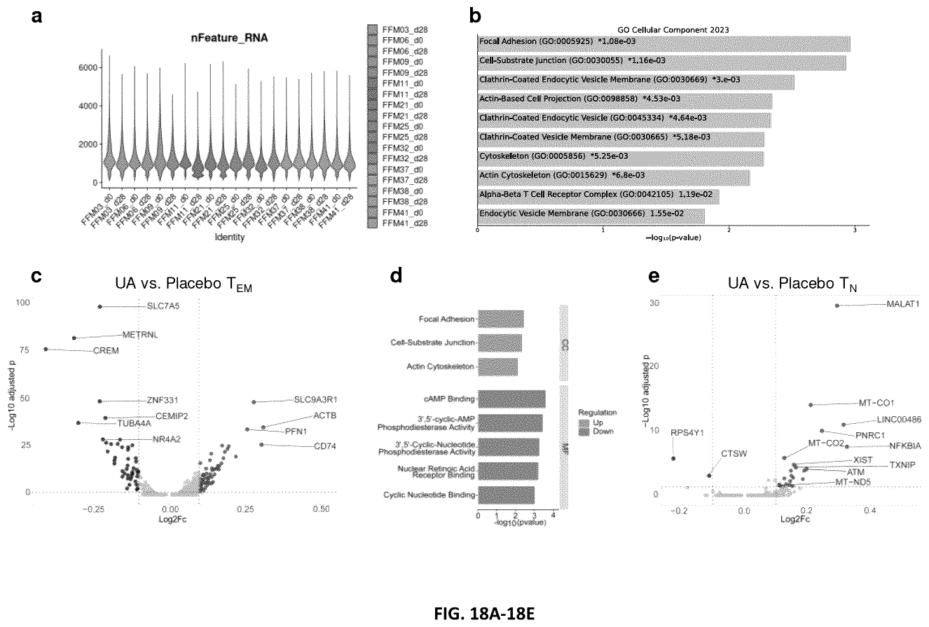

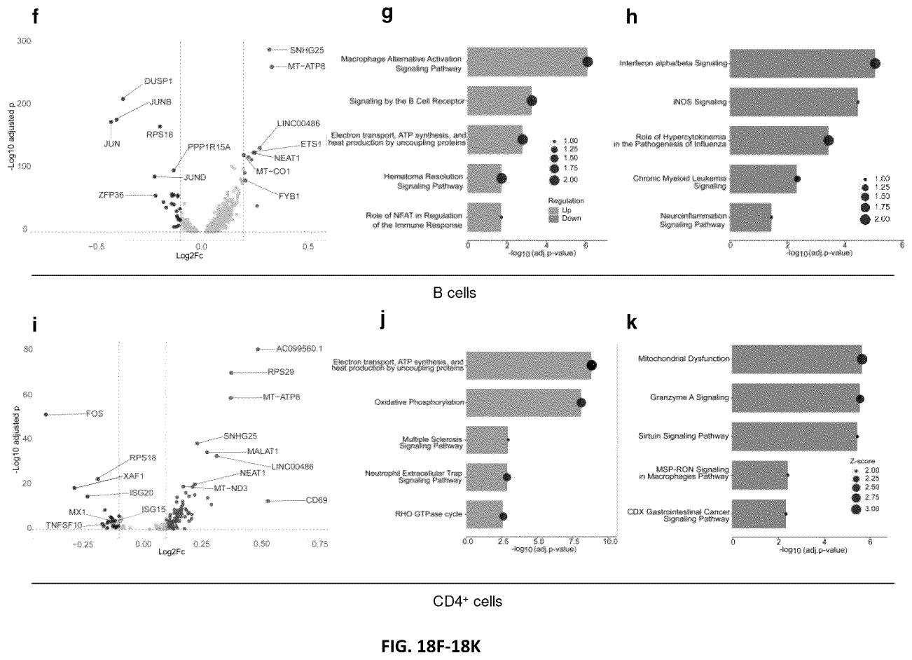

To further profile the immune compartment upon UA intake, we performed single cell (sc) RNA sequencing from five participants before and after both interventions. After quality control filtering, we profiled a total of 231,079 cells with >1000 genes per cell (Extended Data FIG. 17A). For population clustering, a hierarchical approach was used32 incorporating Azimuth52 to define subpopulations (FIG. 6A). A total of 89 genes were differentially expressed in CD8+ T cells (52 up, 37 down) after 28 days of UA compared to placebo (FIG. 16B). Consistent with previous in vitro data29 and our findings of UA eliciting a naïve-like state in T cells (FIG. 3), we found upregulation of the Wnt-associated stemness transcription factors TCF7 and LEF1, as well as induction of IL7R. Among downregulated genes, several genes associated with T cell exhaustion, suppression, and hypofunction (NR4A2, CREM, TGFB1, METRNL) 53-56 were identified. We next sought out to determine pathways affected by UA supplementation. Canonical pathway analysis revealed activation of T cell receptor (TCR) signaling and several pathways associated with cytoskeleton remodeling, adhesion and cellular motility (FIG. 16C) in UA-exposed CD8+ T cells. Likewise, gene ontology enrichment analysis using EnrichR57 revealed a similar tendency towards cytoskeletal reorganization programs (Extended Data FIG. 7B). Among downregulated canonical pathways, both Protein Kinase A (PKA) signaling, and other immune inhibitory pathways were shown to be strongly inhibited (FIG. 16D). Indeed, recent findings suggest G-protein coupled receptors (GPCRs) and Gαs signaling as an immune checkpoint in human cancer that drives a hyporesponsive state, thereby resulting in immunotherapy failure58. Consistent with the notion of reduced PKA signaling upon UA, predicted upstream regulators suggested a reduced role of several components of the GPCR-Gαs-PKA axis, such as GPR174, GNAS and cAMP (FIG. 16E). Notably, other transcription factors central to T cell exhaustion and blunted tumor infiltration such as IRF459, STAT6 and YAP1 were also among predicted inhibited pathways. These findings extended to the T_{EM} subset where we also observed downregulation of the transcription factor ‘cAMP-responsive element modulator’ (CREM; Extended Data FIG. 17C). Gene ontology enrichment analysis confirmed an upregulation of cytoskeletal and adhesion-associated programs, while revealing downregulation of GPCR-Gαs-PKA associated pathways and its ligand PGE2 in TEM as well (Extended Data FIG. 17D). Among naïve T cells, several components of the electron transport chain (MT-ND5, MT-CO1, MT-CO2) were induced upon UA intake (Extended Data FIG. 17E). Collectively, our transcriptomic findings support the notion of altered mitochondrial activity and reduced CD8+ T cell suppression upon UA intake. Changes in the NK cell compartment in UA treated individuals were characterized by reduction of several well-known immediate-early genes (FIG. 16F) including NR4A2, DUSP1, FOS, JUN, and NFKBIA. These constitute primary response genes that are induced rapidly after stimulation and generally do not require de novo protein synthesis. Consistently, other downregulated genes belonged to inflammatory TNF and interferon pathways, while NK cells of UA treated participants also displayed reduction of the activation markers CD69 and CXCR4 (FIG. 16F). Such transcriptional profile is in line with less 12 “inflamed” NK subsets that was recently described60. Furthermore, NK cells also displayed an upregulation of cytoskeletal and adhesion genes. In line with a more mature NK state, upregulated genes included NEAT1 and CD160, thereby reflecting a recently proposed NK1 (B) profile. Consistently, gene ontology enrichment analysis revealed distinct functional and spatial specializations associated with NK1 cells such as cytoskeletal remodeling, adhesion and membrane-protein interactions. In contrast, NK cells of UA receiving participants were less linked to an inflammatory response and nitrogen-associated pathways (FIG. 16G). Monocytes displayed a strong upregulation of several mitochondrial genes (MT-CYB, MT-ND5, MT-ATP8, MT-CO1 among others; FIG. 16H) as well as NAMPT, the rate-limiting enzyme of the NAD salvage pathway that is required to establish an anti-inflammatory M2 polarization. Strongest downregulation was observed among genes that belonged to the interferon response pathway (e.g. IFITM2, IER2, STAT1; FIG. 16I). Interferon-associated and inflammatory pathways were thus predicted as negative upstream regulators whereas inhibitors of pathogenic myeloid-derived inflammation such as ETV3, ETV6 and CITED2 64,65 were assumed to be activated upstream regulators. The transcriptomic characterization therefore reinforces the notion of an anti-inflammatory monocyte polarization under UA treatment. In line with this, downregulated genes by UA in B cells included an inflammatory gene signature (MX1, XAF1, ISG15, TNSF10; Extended Data FIG. 17F) observed in patients with SARS-CoV66. Accordingly, downregulated pathways included hypercytokinemia during viral infections, while B cells displayed upregulation of BCR-associated pathways (Extended Data FIG. 18G-H). CD4+ T cells were largely characterized by a signature associated with mitochondrial health

(Extended Data FIG. 71-K). Taken together, scRNA sequencing data indicated that UA induces a significant change in immune cell transcriptional profiles, with impact on pathways related to inflammation, cellular and mitochondrial metabolism. This is in line with the reduction of immune aging phenotypes described above, further underscoring the UA-dependent potentially beneficial transcriptomic alterations in the human immune system.

### Example 11: Urolithin A Supplementation Reduces Plasma Cyctokines

Considering the effects of UA on immune polarization and the observation that aging-associated metabolic failure profoundly contributes to the phenotype of inflammaging, we next investigated whether circulating cytokine plasma levels are affected by UA supplementation. Administration of UA led to significantly lower levels of several plasma cytokines, namely TNF-α, IL-1β, IFN-γ, IL-2, IL-17 and IL-10 (FIG. 7D-E). No differences were found for IL-6, IL-8, interferon-gamma induced protein (IP-10) and monocyte chemoattractant protein-1 (MCP-1) after UA or placebo intake.

A recent single-cell atlas of healthy human blood has indicated an aging-associated bias towards type 2 immunity that is uncovered in healthy subjects upon antigen challenge, but not at steady-state. We therefore next focused on cytokine expression of stimulated T cells to detect potential differences in the context of immune response which could constitute a potential predisposition to conditions that may only manifest when specifically triggered. To this end, PBMCs from both intervention groups were incubated in the presence of αCD3/αCD28 stimulation beads for a total of four days (FIG. 7C). Intriguingly, challenged CD8+ T cells of UA-treated subjects displayed reduced IL-4 production and enhanced TNF-α production, whereas placebo intake did not elicit any changes (FIG. 7D-E). Taken together, UA treatment resulted in reduced circulating pro-inflammatory cytokines at steady-state and an antigen-provoked tendency towards a type 1 response.

The immunosenescent phenotype is considered responsible for an increased susceptibility to infections. Given the established role of UA in instructing macrophage polarization in vitro67 in addition to here suggesting altered circulating monocyte composition, we lastly intended to further characterize their functional capacities upon UA intake. For this, we performed a phagocytosis assay to assess whether UA impacts monocyte phagocytotic capabilities of gram-negative bacteria. Monocytes from UA-exposed participants exhibited 14 significantly enhanced phagocytosis of E. coli particles ex vivo when compared with the placebo cohort (FIG. 17F), hinting at superior uptake and clearance of gram-negative bacteria.

Collectively, our data supports the notion that apart from newly characterized profound phenotypic, metabolic and transcriptomic changes in the human immune system, UA supplementation results in changes in immune function that potentially carries systemic consequences. Considering the effects of UA on immune polarization and the observation that aging-associated metabolic failure profoundly contributes to the phenotype of inflammaging, we next investigated whether circulating cytokine plasma levels are affected by UA supplementation. Administration of UA led to significantly lower levels of several plasma cytokines, namely TNF-α, IL-1β, IFN-γ, IL-2, IL-17 and IL-10 (FIG. 7D-E). No differences were found for IL-6, IL-8, interferon-gamma induced protein (IP-10) and monocyte chemoattractant protein-1 (MCP-1) after UA or placebo intake.

A recent single-cell atlas of healthy human blood has indicated an aging-associated bias towards type 2 immunity that is uncovered in healthy subjects upon antigen challenge, but not at steady-state. We therefore next focused on cytokine expression of stimulated T cells to detect potential differences in the context of immune response which could constitute a potential predisposition to conditions that may only manifest when specifically triggered. To this end, PBMCs from both intervention groups were incubated in the presence of αCD3/αCD28 stimulation beads for a total of four days (FIG. 17C). Intriguingly, challenged CD8+ T cells of UA-treated subjects displayed reduced IL-4 production and enhanced TNF-α production, whereas placebo intake did not elicit any changes (FIG. 17D-E). Taken together, UA treatment resulted in reduced circulating pro-inflammatory cytokines at steady-state and an antigen-provoked tendency towards a type 1 response.

The immunosenescent phenotype is considered responsible for an increased susceptibility to infections. Given the established role of UA in instructing macrophage polarization in vitro67 in addition to here suggesting altered circulating monocyte composition, we lastly intended to further characterize their functional capacities upon UA intake. For this, we performed a phagocytosis assay to assess whether UA impacts monocyte phagocytotic capabilities of gram-negative bacteria. Monocytes from UA-exposed participants exhibited 14 significantly enhanced phagocytosis of E. coli particles ex vivo when compared with the placebo cohort (FIG. 17F), hinting at superior uptake and clearance of gram-negative bacteria.

Collectively, our data supports the notion that apart from newly characterized profound phenotypic, metabolic and transcriptomic changes in the human immune system, UA supplementation results in changes in immune function that potentially carries systemic consequences.

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

1. A method of ameliorating immune aging, comprising administering to a human subject in need thereof an effective amount of a compound of formula (I), or a salt, prodrug, metabolite or derivative thereof; 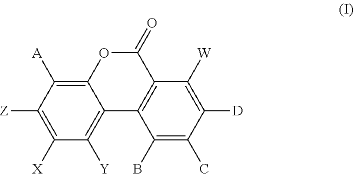
wherein:
A, B, C, D, W, X, Y and Z are each independently selected from H and OH.

2. The method of claim 1, wherein the amelioration of immune aging comprises one or more of:
an increase in the ratio of CD8+ (T_{N}) T cells to CD8+ (T_{EM}) T cells so that the proportion of naïve CD8+ T cells is increased;
an increase in the proportion of CD56^{dim}CD16^{bright} NK (Natural Killer) cells in the total PBMC population;
an increase in mitochondrial function in immune cells;
a decrease in inflammatory markers; and
an increase in circulating lymphocyte count.

3. The method of claim 1, wherein the human subject is over 40 years of age, for example over 45 years of age, 50 years of age, for example over 55 years of age, for example over 60 years of age, for example over 65 years of age, for example over 70 years or age, for example over 75 years of age, for example over 80 years of age, for example over 85 years of age.

4. The method of claim 1, wherein the human subject is one who has been identified as having one or more of:
a ratio of CD8+ (T_{N}) T cells to CD8+ (T_{EM}) T cells that is lower than needed for strong immune health;
a proportion of CD56^{dim}CD16^{bright} NK (Natural Killer) cells is lower than needed for strong immune health;
mitochondrial function in immune cells that is lower than needed for strong immune health;
inflammatory markers indicative of significant inflammaging; and
a circulating lymphocyte count that is lower than needed for strong immune health.

5. The method of claim 1, wherein the human subject presents with an IMM-AGE score indicating a higher mortality or immune age than the average for a subject of the same age.

6. The method of claim 1, wherein the human subject presents with a ratio of naïve CD8+ T cells to effector memory CD8+ T cells that is biased towards memory CD8+ T cells.

7. The method of claim 1, wherein the human subject is displaying signs or symptoms of inflammaging.

8. The method of claim 1, wherein the human subject has cancer and is undergoing a cancer therapy, for example a cancer immunotherapy, and has one or more of the following as a result of the therapy:
a ratio of CD8+ (T_{N}) T cells to CD8+ (T_{EM}) T cells that is lower than before the therapy started;
a proportion of CD56^{dim}CD16^{bright} NK (Natural Killer) cells is lower than before the therapy started;
mitochondrial function in immune cells that is lower than before the therapy started; and
a circulating lymphocyte count that is lower than before the therapy started.

9. A method of reducing biological age, comprising administering to a human subject in need thereof an effective amount of a compound of formula (I), or a salt, prodrug, metabolite or derivative thereof; 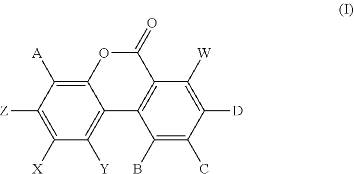
wherein:
A, B, C, D, W, X, Y and Z are each independently selected from H and OH.

10. The method of claim 9, wherein the biological age is biological immune age or a biological age specific to a target organ, for example skin biological age, brain biological age or muscle biological age.

11. The method of claim 9, wherein the biological age is biological age of immune cells, for example, biological age of peripheral blood mononuclear cells.

12. (canceled)

13. (canceled)

14. (canceled)

15. (canceled)

16. (canceled)

17. (canceled)

18. (canceled)

19. (canceled)

20. The method of claim 1, wherein the compound is administered orally, topically or by inhalation.

21. The method of claim 20, wherein the compound is comprised by a dietary supplement or a medical food.

22. The method of claim 20, wherein the compound is comprised by a pharmaceutical composition.

23. The method of claim 1, further comprising administering a further pharmaceutical compound.

24. The method of claim 1, wherein the further pharmaceutical compound is selected from an anti-cancer compound, an antibiotic compound, and an anti-viral compound.

25. A method of analysing a drug, dietary supplement, medical food or other treatment of a human subject to establish whether the treatment is exerting its effects by improving mitochondrial health (for example mitochondrial immune health), to establish whether the agent is being efficacious, or to establish an optimised dose of the treatment comprising:
analysing a blood sample taken before the treatment and comparing it with a blood sample taken after the treatment has taken place; and
carrying out flow cytometry to analyse single circulating cells (for example circulating immune; for example T cells, NK cells or other monocytes);
analysing whether there has been a change in mitochondrial mass, mitochondrial membrane potential or mitochondrial biogenesis; or
a method of assessing whether a drug or other treatment of a human subject is exerting its effects by improving mitochondrial health comprising analysing a blood sample taken from the subject before and after treatment and carrying out flow cytometry to analyse single circulating cells (for example circulating immune cells; for example T cells, NK cells or other monocytes), for example, using SCINETH, and analysing one or more of the following:
(1) change in mitochondrial mass (for example as analysed by staining with a cationic fluorophore that accumulates electrophoretically in mitochondria, for example a dye available under the name MitoTracker Green),
(2) change in mitochondrial membrane potential (for example as analysed by staining with a dye that is responsive to the membrane potential for example a dye available under the name MitoTracker Red), and/or
(3) change in mitochondrial biogenesis (for example as anaylsed by assessing the level of PGC alpha).

26. A method of preparing a CAR-T cell preparation, wherein said method comprises the steps of:
(a) optionally treating a patient with one or more mitophagy inducers (for example, urolithin A), for example, treating a patient for about 1 month with the one or more mitophagy inducers;
(b) obtaining a sample comprising T-cells from the patient, for example, obtaining a blood sample;
(c) isolating T-cells from the sample;
(d) Transfecting the T cells with a CAR (chimeric antigen receptor) gene, to prepare CAR-T cells; and
(e) expanding the number of CAR-T cells, for example, in media containing one or more mitophagy inducers (for example, urolithin A), to prepare a CAR-T cell preparation; or
a method of measuring immune health comprising analysing one or more of the following:
(1) change in mitochondrial mass (for example as analysed by staining with a cationic fluorophore that accumulates electrophoretically in mitochondria, for example a dye available under the name MitoTracker Green),
(2) change in mitochondrial membrane potential (for example as analysed by staining with a dye that is responsive to the membrane potential for example a dye available under the name MitoTracker Red), and/or
(3) change in mitochondrial biogenesis (for example as anaylsed by assessing the level of PGC alpha).

27. (canceled)

28. (canceled)

29. The method of claim 1, wherein the human subject is a healthy human subject.

30. The method of claim 9, wherein the human subject is a healthy human subject; and the reduction in biological age is assessed based the degree of DNA methylation in the subject or based on the degree of methylation of DNA in a sample of circulating PBMCs obtained from the subject.
